(19) United States
(12) Patent Application Publication
(10) Pub. No.: US 2021/0157038 A1
(43) Pub. Date: May 27, 2021

(54) PRISM-COUPLING SYSTEMS AND METHODS HAVING MULTIPLE LIGHT SOURCES WITH DIFFERENT WAVELENGTHS

(71) Applicant: CORNING INCORPORATED, CORNING, NY (US)

(72) Inventors: Ryan Claude Andrews, Elmira, NY (US); Pierre Michel Bouzi, Horseheads, NY (US); Zhenhua Guo, Painted Post, NY (US); Jacob Immerman, Coming, NY (US); Jeremiah Robert Jacobson, Corning, NY (US); Michael David Moon, Cameron Mills, NY (US); Babak Robert Raj, Corning, NY (US); Nathaniel David Wetmore, Corning, NY (US); Xiupu Wang, KunShan City (CN)

(21) Appl. No.: 17/104,154

(22) Filed: Nov. 25, 2020

Related U.S. Application Data
(60) Provisional application No. 62/940,295, filed on Nov. 26, 2019.

(51) Int. Cl. G02B 5/04; G01N 21/41; G01L 1/24; G02B 6/14

(52) U.S. Cl. CPC G02B 5/04; G02B 6/14; G01L 1/24; G01N 21/41

(57) ABSTRACT

The prism-coupling systems and methods include using a prism-coupling system to collect initial TM and TE mode spectra of a chemically strengthened article having a refractive index profile with a near-surface spike region and a deep region. The prism-coupling system has a light source configured to generate sequential measurement light beams or reflected light beams having different measurement wavelengths. The different measurement wavelengths generate different TM and TE mode spectra. The light source can include multiple light-emitting elements and optical filters or a broadband light source and optical filters. The optical filters can be sequentially inserted into either the input optical path or the output optical path of the prism-coupling system.

This application claims the benefit of priority of U.S. Provisional Application Ser. No. 62/940,295 filed on Nov. 26, 2019 the content of which is relied upon and incorporated herein by reference in its entirety.

The present disclosure relates to prism-coupling systems and methods used for characterizing stress in glass-based chemically strengthened articles, and in particular relates to such systems and methods having multiple light sources with different wavelengths.

### BACKGROUND

Chemically strengthened glass-based articles are formed by subjecting glass-based substrates to a chemical modification to improve at least one strength-related characteristic, such as hardness, resistance to fracture or surface scratches, etc. Chemically strengthened glass-based articles have found particular use as cover glasses for display-based electronic devices, especially hand-held devices such as smart phones and tablets.

In one method, the chemical strengthening is achieved by an ion-exchange (IOX) process whereby ions in the matrix of a glass-based substrate (“native ions” or “substrate ions”) are replaced by externally introduced (i.e., replacement or in-diffused) ions, e.g., from a molten bath. The strengthening generally occurs when the replacement ions are larger than the native ions (e.g., Na⁺ or Li⁺ ions replaced by K⁺ ions). The IOX process gives rise to an IOX region in the glass that extends from the article surface into the matrix. The IOX region defines within the matrix a refractive index profile having a depth of layer (DOL) that represents a size, thickness or “deepness” of the IOX region as measured relative to the article surface. The refractive index profile also defines stress-related characteristics, including a stress profile, surface stress, depth of compression, center tension, birefringence, etc. The refractive index profile can also define in the glass-based article an optical waveguide that supports a number m of guided modes for light of a given wavelength when the refractive index profile meets certain criteria known in the art.

Prism-coupling systems and methods can be used to measure the spectrum of the guided modes of the planar optical waveguide formed in the glass-based IOX article to characterize one or more properties of the IOX region, such as the refractive index profile and the aforementioned stress-related characteristics. This technique has been used to measure properties of glass-based IOX articles used for a variety of applications, such as for chemically strengthened covers for displays (e.g., for smart phones). Such measurements are used for quality control purposes to ensure that the IOX region has the intended characteristics and falls within the select design tolerances for each of the selected characteristics for the given application.

While prism-coupling systems and methods can be used for many types of conventional glass-based IOX articles, such methods do not work as well and sometimes do not work at all on certain glass-based IOX articles. For example, certain types of IOX glass-based articles are actual dual IOX (DIOX) glass-based articles formed by first and second ion diffusions that give rise to a two-part stress profile. The first part (first region) is immediately adjacent the substrate surface and has a relatively steep slope for the stress change, while the second segment (second region) extends deeper into the substrate but has a relatively shallow slope for the stress change. The first region is referred to as the spike region or just “spike,” while the second region is referred to as the deep region. The optical waveguide is defined by both the spike region and the deep region.

Such two-region profiles result in a relatively large spacing between low-order modes, which have a relatively high effective index, and a very small spacing between high-order modes, which have a relatively low effective index close to the critical angle, which defines the boundary or transition between total-internal reflection (TIR) for guided modes and non-TIR for so-called leaky modes. In a mode spectrum, the critical angle can also be called the “critical angle transition” for convenience. It can happen that a guided mode can travel only in the spike region of the optical waveguide. A guided or leaky mode traveling only in the spike region makes it difficult if not impossible to distinguish between light that is guided only in the spike region and light that is guided in the deep region.

Determining the precise location of the critical angle from the mode spectrum for a glass-based IOX article having a two-region profile is problematic because guided modes that reside close to the critical angle distort the intensity profile at the critical angle transition. This in turn distorts the calculation of the fractional number of mode fringes, and hence the calculation of the depth of the spike region and stress-related parameters, including the calculation of the compressive stress at the bottom of the spike region, which is referred to as the “knee stress” and is denoted CSₖ.

As it turns out, the knee stress CSₖ is an important property of a glass-based IOX article and its measurement can be used for quality control in large-scale manufacturing of chemically strengthened glass-based articles. Unfortunately, the above-described measurement problems impose severe restrictions when using a prism-coupling system to make measurements of IOX articles for quality control because an accurate estimation of the knee stress CSₖ requires that the critical angle transition be accurately established for both the transverse electric (TE) and transverse magnetic (TM) guided modes.

### SUMMARY

The methods described herein are directed to improving the performance of a prism-coupling system when measuring at least one stress-related characteristic of chemically strengthened articles, and particularly for IOX articles that include a near-surface spike region. The improvement includes a light source comprising multiple light-emitting elements having different measurement wavelengths or alternatively a single wideband light source and multiple narrow-band filters to define the different measurement wavelengths. Measuring chemically strengthened articles at different wavelengths allows for a more accurate estimate of at least one stress-related characteristic. Example stress-related characteristics include stress-related parameters, such as the stress profile, the knee stress CSₖ, the center tension CT, the tension-strain energy TSE, birefringence, and an estimate of frangibility, which relates to the center tension CT and/or to the tension-strain energy TSE, the spike depth D1, the depth of layer D2 and the refractive index profile n(x).

Examples of the prism-coupling systems and methods include using a prism-coupling system to collect initial TM and TE mode spectra of a chemically strengthened article. In an example, the chemically strengthened article has a refractive index profile with a near-surface spike region and a deep region. TM and TE mode spectra are collected sequentially at two or more different measurement wavelengths, i.e., the different emission wavelengths of the multiple light-emitting elements of the light source or the different measurement wavelengths formed by filtering the wide-band light from a wide-band light source. This results in a set of TM and TE mode spectra for the different measurement wavelengths. The set of TM and TE mode spectra is then evaluated to assess which of the TM and TM mode spectra is best suited for determining at least one stress characteristic. The evaluation can include considering the contrast of the mode lines in the TM and TE mode spectra. The evaluation can also include determining integer and fractional parts of the number of mode lines in the TM mode spectrum and TE mode spectrum and making a selection based on the fractional part (FP) falling into a select range or having a select value, as explained below.

An embodiment of the disclosure is directed to a method of estimating a least one stress-based characteristic of a chemically strengthened article having a refractive index profile with a near-surface spike region and a deep region that define an optical waveguide in a glass-based substrate, comprising: a) using a prism-coupling system having a light source and a coupling prism, sequentially illuminating the glass-based substrate through the coupling prism with measurement light of different wavelengths to generate reflected light containing TM and TE mode spectra for each measurement wavelength to define a set of TM and TE mode spectra; b) examining the set of TM and TE mode spectra to identify a best TM and TE mode spectra of the set of TM and TE mode spectra for providing a most accurate estimate of the at least one stress-based characteristic; and c) estimating the at least one stress-based characteristic using the best TM and TE mode spectra.

Another embodiment of the disclosure is directed to a method of estimating a least one stress-based characteristic of a chemically strengthened article having a refractive index profile with a near-surface spike region and a deep region that define an optical waveguide in a glass-based substrate, comprising: a) using a prism-coupling system having a light source and a coupling prism, sequentially illuminating the glass-based substrate through the coupling prism with broadband measurement light to generate broadband reflected light containing TM and TE mode spectra; b) sequentially narrow-band filtering either the broadband measurement light or the broadband reflected light to form sequential narrow-band reflected light beams having different center wavelengths; c) digitally detecting the sequential narrow-band reflected light beams to capture TM and TE mode spectra for each of the sequential narrow-band reflected light beams; d) examining the set of TM and TE mode spectra to identify a best TM and TE mode spectra of the set of TM and TE mode spectra for providing a most accurate estimate of the at least one stress-based characteristic; and e) estimating the at least one stress-based characteristic using the best TM and TE mode spectra.

Another embodiment of the disclosure is directed to a prism-coupling system for measuring a stress characteristic of a chemically strengthened ion-exchanged (IOX) article having a near-surface spike region and a deep region formed in a glass-based substrate and that define an optical waveguide, comprising: a) a coupling prism having an input surface, an output surface and a coupling surface, and wherein the coupling surface interfaces with the waveguide at a substrate upper surface; b) a light source system that sequentially emits over an input optical path multiple measurement light beams having different measurement wavelengths, wherein the sequentially emitted measurement light beams illuminate the interface through the input surface of the prism, thereby forming sequentially reflected light beams that exit the output surface of the coupling prism and travel over an output optical path, wherein the sequentially reflected light beams defines respective transverse magnetic (TM) mode spectrum and a transverse electric (TE) mode spectrum each having a different one of the measurement wavelengths; c) a photodetector system arranged to receive the sequentially reflected light beams and detect the TM and TE mode spectra for each of the measurement wavelengths to form a set of TM and TE mode spectra; d) a controller configured to perform the acts of: i) processing the set of TM and TE mode spectra to identify a best TM and TE mode spectra of the set of TM and TE mode spectra for providing a most accurate estimate of the at least one stress-based characteristic; and ii) estimating the at least one stress-based characteristic using the best TM and TE mode spectra.

Another embodiment of the disclosure is directed to a prism-coupling system for measuring a stress characteristic of a chemically strengthened ion-exchanged (IOX) article having a near-surface spike region and a deep region formed in a glass-based substrate and that define an optical waveguide, comprising: a) a coupling prism having an input surface, an output surface and a coupling surface, and wherein the coupling surface interfaces with the waveguide at a substrate upper surface; b) a light source system that sequentially emits over an input optical path a broadband light beam that illuminates the interface through the input surface of the prism, thereby forming a reflected light beam that exit the output surface of the coupling prism and travel over an output optical path, wherein the reflected light beam defines a transverse magnetic (TM) mode spectrum and a transverse electric (TE) mode spectrum; c) an optical filter system configured to sequentially insert optical filters having different narrow-band wavelength transmissions into either the input optical path or the output optical path to define sequentially reflected light beams each having a different measurement wavelength; d) a photodetector system arranged to receive the sequential reflected light beams and detect the TM and TE mode spectra for each of the measurement wavelengths to form a set of TM and TE mode spectra; and e) a controller configured to perform the acts of: i) identifying a best TM and TE mode spectra of the set of TM and TE mode spectra for providing a most accurate estimate of the at least one stress-based characteristic; and ii) estimating the at least one stress-based characteristic using the best TM and TE mode spectra.

According to aspect (1), a method of estimating a least one stress-based characteristic of a chemically strengthened article having a refractive index profile with a near-surface spike region and a deep region that define an optical waveguide in a glass-based substrate is provided. The method comprises: a) using a prism-coupling system having a light source and a coupling prism, sequentially illuminating the glass-based substrate through the coupling prism with measurement light of different wavelengths to generate reflected light containing TM and TE mode spectra for each measurement wavelength to define a set of TM and TE mode spectra; b) examining the set of TM and TE mode spectra and identifying a best TM and TE mode spectra of the set of TM and TE mode spectra for providing a most accurate estimate of the at least one stress-based characteristic; and c) estimating the at least one stress-based characteristic using the best TM and TE mode spectra.

According to aspect (2), the method of aspect (1) is provided, wherein the at least one stress-related characteristic comprises at least one of: a stress profile, a knee stress, a center tension, a tension-strain energy, a birefringence, a frangibility, a spike depth, a depth of layer, and a refractive index profile.

According to aspect (3), the method of any of aspect (1) to the preceding aspect is provided, wherein each of the TM and TE mode spectra has fringes with a fringe contrast, a critical transition and a fringe count with an integer part and a fractional part FP, and wherein identifying a best TM and TE mode spectra of the set of TM and TE mode spectra comprises at least one of: selecting the TM and TE mode spectra having the greatest fringe contrast; selecting the TM and TE mode spectra having respective fractional parts FP in a range between 0.1 and 0.85; and selecting the TM and TE mode spectra where the respective fringes are least affected by the respective critical transitions.

According to aspect (4), the method of aspect (3) is provided, wherein the fractional part FP is between 0.15 and 0.8.

According to aspect (5), the method of any of aspect (1) to the preceding aspect is provided, wherein the different measurement wavelengths fall within a wavelength range from 350 nm to 850 nm.

According to aspect (6), the method of aspect (5) is provided, wherein the different measurement wavelengths fall within a wavelength range from 540 nm to 650 nm.

According to aspect (7), the method of any of aspect (1) to the preceding aspect is provided, wherein the prism-coupling system comprises a light source comprising multiple light-emitting elements, wherein each of light-emitting elements emits light at one of the different measurement wavelengths, and wherein changing the measurement configuration includes translating the light source device so that the multiple light-emitting devices are sequentially aligned with an input optical axis that runs between the light source and a coupling prism.

According to aspect (8), the method of aspect (7) is provided, wherein the multiple light-emitting elements comprise light-emitting diodes or laser diodes.

According to aspect (9), the method of any of aspect (7) to the preceding aspect is provided, wherein the different wavelengths of the different light-emitting elements differ by between 1% and 25%.

According to aspect (10), the method of aspect (9) is provided, wherein the different wavelengths of the different light-emitting elements differ by between 3% and 11%.

According to aspect (11), the method of any of aspect (1) to the preceding aspect is provided, wherein the light source device is mechanically connected to a motion control system, and wherein said translating of the light source device is carried out by activating the motion control system.

According to aspect (12), the method of aspect (11) is provided, wherein the motion control comprises a linear actuator.

According to aspect (13), the method of any of aspect (1) to the preceding aspect is provided, wherein the measurement light from each of the light-emitting elements has a wavelength bandwidth centered around a central wavelength, and further comprising sequentially passing the measurement light of the different wavelengths through respective narrow-pass optical filters centered on the respective different central wavelengths to reduce the wavelength bandwidth of the measurement light.

According to aspect (14), the method of any of aspects (1) to (6) is provided, wherein the prism-coupling system comprises a light source comprising a broadband light-emitting element that emits broadband light, and wherein changing the measurement configuration includes sequentially filtering the broadband light with two or more narrow-band optical filters centered on different measurement wavelengths.

According to aspect (15), the method of aspect (14) is provided, wherein the light-emitting element comprises multiple light emitters.

According to aspect (16), the method of any of aspect (14) to the preceding aspect is provided, wherein the two or more narrow-band optical filters are supported in a filter member and further comprising moving the filter member to sequentially place the narrow-band optical filters either in operable alignment with the broadband light-emitting element or within the reflected light.

According to aspect (17), the method of aspect (16) is provided, wherein the filter member comprises a filter wheel and said moving of the filter member comprises rotating the filter member.

According to aspect (18), the method of any of aspect (16) to the preceding aspect is provided, further comprising tracking a position of the filter member using a detection system to ensure alignment of a select one of the narrow-band optical filters either with the broadband light-emitting element or within the reflected light.

According to aspect (19), the method of any of aspects (14) to (16) is provided, wherein the two or more narrow-band optical filters are supported by a support frame and linearly translating the support frame to sequentially place the narrow-band optical filters in operable alignment with the broadband light-emitting element.

According to aspect (20), the method of aspect (19) is provided, wherein the linearly translating of the support frame is performed by activating a motion control system mechanically coupled to the support frame.

According to aspect (21), the method of aspect (20) is provided, wherein the motion control system comprises a linear actuator and wherein said linearly translating comprises activating the linear actuator.

According to aspect (22), the method of any of aspect (1) to the preceding aspect is provided, wherein the examining of the set of TM and TE mode spectra comprises detecting each of the TM and TE mode spectra with a digital detector and digitally processing respective mode lines of the TM and TE mode spectra to establish a mode line contrast.

According to aspect (23), the method of any of aspect (1) to the preceding aspect is provided, comprising optically coupling the coupling prism to the chemically strengthened article by an index matching fluid.

According to aspect (24), a method of estimating a least one stress-based characteristic of a chemically strengthened article having a refractive index profile with a near-surface spike region and a deep region that define an optical waveguide in a glass-based substrate is provided. The method comprises: a) using a prism-coupling system having a light source and a coupling prism, sequentially illuminating the glass-based substrate through the coupling prism with broadband measurement light to generate broadband reflected light containing TM and TE mode spectra; b) sequentially narrow-band filtering either the broadband measurement light or the broadband reflected light to form sequential narrow-band reflected light beams having different center wavelengths; c) digitally detecting the sequential narrow-band reflected light beams to capture TM and TE mode spectra for each of the sequential narrow-band reflected light beams; d) examining the set of TM and TE mode spectra to identify a best TM and TE mode spectra of the set of TM and TE mode spectra for providing a most accurate estimate of the at least one stress-based characteristic; and e) estimating the at least one stress-based characteristic using the best TM and TE mode spectra.

According to aspect (25), the method of aspect (24) is provided, wherein the at least one stress-related characteristic comprises at least one of: a stress profile, a knee stress, a center tension, a tension-strain energy, a birefringence, a frangibility, a spike depth, a depth of layer, and a refractive index profile.

According to aspect (26), the method of any of aspect (24) to the preceding aspect is provided, wherein each of the TM and TE mode spectra has fringes with a fringe contrast, a critical transition and a fringe count with an integer part and a fractional part FP, and wherein identifying a best TM and TE mode spectra of the set of TM and TE mode spectra comprises at least one of: selecting the TM and TE mode spectra having the greatest fringe contrast; selecting the TM and TE mode spectra having respective fractional parts FP in a range between 0.1 and 0.85; and selecting the TM and TE mode spectra where the respective fringes are least affected by the respective critical transitions.

According to aspect (27), the method of aspect (26) is provided, wherein the fractional part FP is between 0.15 and 0.8.

According to aspect (28), the method of any of aspect (24) to the preceding aspect is provided, wherein the different measurement wavelengths fall within a wavelength range from 350 nm to 850 nm.

According to aspect (29), the method of aspect (28) is provided, wherein the different measurement wavelengths fall within a wavelength range from 540 nm to 650 nm.

According to aspect (30), the method of any of aspect (24) to the preceding aspect is provided, wherein the different wavelengths of the different light-emitting elements differ by between 1% and 25%.

According to aspect (31), the method of aspect (30) is provided, wherein the different wavelengths of the different light-emitting elements differ by between 2% and 15%.

According to aspect (32), the method of aspect (31) is provided, wherein the different wavelengths of the different light-emitting elements differ by between 3% and 11%.

According to aspect (33), the method of any of aspect (24) to the preceding aspect is provided, wherein the narrow band filtering comprises sequentially inserting narrow band filters having the different center wavelengths into either the broadband measurement light or the broadband reflected light.

According to aspect (34), the method of aspect (33) is provided, wherein the narrow band filters are supported in a filter member and wherein act of sequentially inserting comprises moving the filter member.

According to aspect (35), the method of aspect (34) is provided, wherein the filter member comprises a filter wheel and wherein said moving the filter member comprises rotating the filter wheel.

According to aspect (36), the method of any of aspect (34) to the preceding aspect is provided, further comprising tracking a position of the filter member using a detection system.

According to aspect (37), the method of any of aspects (34) to (36) is provided, wherein said digitally detecting comprises focusing the sequential narrow-band reflected light beams onto a digital detector using a collection optical system, and wherein the filter member at least partially resides within the collection optical system.

According to aspect (38), the method of any of aspects (34) to (36) is provided, wherein the said digitally detecting comprises focusing the sequential narrow-band reflected light beams onto a digital detector using a collection optical system, and wherein the filter member resides between the coupling prism and the collection optical system.

According to aspect (39), the method of any of aspect (24) to the preceding aspect is provided, wherein the sequential narrow-band reflected light beams each has a wavelength band of 10 nm or less.

According to aspect (40), the method of aspect (39) is provided, wherein the sequential narrow-band reflected light beams each has a wavelength band of 6 nm or less.

According to aspect (41), a prism-coupling system for measuring a stress characteristic of a chemically strengthened ion-exchanged (IOX) article having a near-surface spike region and a deep region formed in a glass-based substrate and that define an optical waveguide is provided. The prism-coupling system comprising: a) a coupling prism having an input surface, an output surface and a coupling surface, and wherein the coupling surface interfaces with the waveguide at a substrate upper surface; b) a light source system that sequentially emits over an input optical path multiple measurement light beams having different measurement wavelengths, wherein the sequentially emitted measurement light beams illuminate the interface through the input surface of the prism, thereby forming sequentially reflected light beams that exit the output surface of the coupling prism and travel over an output optical path, wherein the sequentially reflected light beams defines respective transverse magnetic (TM) mode spectrum and a transverse electric (TE) mode spectrum each having a different one of the measurement wavelengths; c) a photodetector system arranged to receive the sequentially reflected light beams and detect the TM and TE mode spectra for each of the measurement wavelengths to form a set of TM and TE mode spectra, d) a controller configured to perform the acts of: a. processing the set of TM and TE mode spectra to identify a best TM and TE mode spectra of the set of TM and TE mode spectra for providing a most accurate estimate of the at least one stress-based characteristic; and b. estimating the at least one stress-based characteristic using the best TM and TE mode spectra.

According to aspect (42), the prism-coupling system of aspect (41) is provided, wherein the light source system comprises a light source device that operably supports multiple light-emitting elements having different measurement wavelengths.

According to aspect (43), the prism-coupling system of aspect (42) is provided, wherein the light source device is mechanically connected to a motion control system that moves the light source device so that the light-emitting elements sequentially emit measurement light of the different measurement wavelengths over the input optical path.

According to aspect (44), the prism-coupling system of aspect (43) is provided, wherein the motion control system comprises a linear actuator connected to the light source by a drive shaft.

According to aspect (45), the prism-coupling system of any of aspect (42) to the preceding aspect is provided, wherein the sequentially emitted measurement light beams each has an optical bandwidth and further comprising multiple narrow-band optical filters, wherein the optical filters are supported in a support frame so that each of the light-emitting elements of the multiple light-emitting elements is optically aligned with one of the optical filters to reduce the optical bandwidth of the sequentially emitted measurement light beams.

According to aspect (46), the prism-coupling system of aspect (41) is provided, wherein the light source comprises a broadband light-emitting element that emits broadband light, and further comprising an array of optical filters each having a different central wavelength, wherein the optical filters are supported by a movable support frame so that the optical filters can be sequentially inserted into the broadband light to generate the sequential measurement light beams having the different measurement wavelengths.

According to aspect (47), the prism-coupling system of aspect (46) is provided, wherein the movable support frame comprises a rotatable filter member.

According to aspect (48), the prism-coupling system of aspect (46) is provided, wherein the movable support frame is operably attached to a linear actuator configured to translate the support frame to sequentially insert the optical filters into the broadband light.

According to aspect (49), the prism-coupling system of any of aspect (41) to the preceding aspect is provided, wherein the sequentially emitted measurement light beams each has an optical bandwidth of less than 10 nm.

According to aspect (50), the prism-coupling system of any of aspect (41) to the preceding aspect is provided, wherein the different measurement wavelengths consist of three different measurement wavelengths.

According to aspect (51), the prism-coupling system of any of aspect (41) to the preceding aspect is provided, wherein the different measurement wavelengths fall within the wavelength range from 350 nm to 850 nm.

According to aspect (52), the prism-coupling system of aspect (51) is provided, wherein the different measurement wavelengths fall within a wavelength range from 540 nm to 650 nm.

According to aspect (53), a prism-coupling system for measuring a stress characteristic of a chemically strengthened ion-exchanged (IOX) article having a near-surface spike region and a deep region formed in a glass-based substrate and that define an optical waveguide is provided. The prism-coupling system comprises: a) a coupling prism having an input surface, an output surface and a coupling surface, and wherein the coupling surface interfaces with the waveguide at a substrate upper surface; b) a light source system that sequentially emits over an input optical path a broadband light beam that illuminates the interface through the input surface of the prism, thereby forming a reflected light beam that exit the output surface of the coupling prism and travel over an output optical path, wherein the reflected light beam defines a transverse magnetic (TM) mode spectrum and a transverse electric (TE) mode spectrum; c) an optical filter system configured to sequentially insert optical filters having different narrow-band wavelength transmissions into either the input optical path or the output optical path to define sequentially reflected light beams each having a different measurement wavelength; d) a photodetector system arranged to receive the sequential reflected light beams and detect the TM and TE mode spectra for each of the measurement wavelengths to form a set of TM and TE mode spectra; e) a controller configured to perform the acts of: a. identifying a best TM and TE mode spectra of the set of TM and TE mode spectra for providing a most accurate estimate of the at least one stress-based characteristic; and b. estimating the at least one stress-based characteristic using the best TM and TE mode spectra.

According to aspect (54), the prism-coupling system of aspect (53) is provided, wherein the optical filter system comprises a movable filter member mechanically connected to a drive motor configured to move the movable filter member.

According to aspect (55), the prism-coupling system of aspect (54) is provided, further comprising a detection system for detecting a position of the movable filter member.

According to aspect (56), the prism-coupling system of any of aspect (54) to the preceding claim is provided, wherein the moveable filter member comprises a rotatable filter wheel.

According to aspect (57), the prism-coupling system of any of aspect (53) to the preceding claim is provided, wherein said identifying the best TM and TE mode spectra comprises performing a fringe count to determine an integer part of the fringe count and a fractional part of the fringe count and selecting the TM and TE mode spectra based on the fractional part of the fringe count falling within a select range.

According to aspect (58), the prism-coupling system of any of aspect (53) to the preceding claim is provided, wherein the sequentially reflected light beams each has a wavelength band of less than 10 nm.

According to aspect (59), the prism-coupling system of any of aspect (53) to the preceding claim is provided, wherein the different measurement wavelengths fall within the wavelength range from 350 nm to 850 nm.

According to aspect (60), the prism-coupling system of aspect (59) is provided, wherein the different measurement wavelengths fall within a wavelength range from 540 nm to 650 nm.

Additional features and advantages are set forth in the Detailed Description that follows, and in part will be apparent to those skilled in the art from the description or recognized by practicing the embodiments as described in the written description and claims hereof, as well as the appended drawings. It is to be understood that both the foregoing general description and the following Detailed Description are merely exemplary, and are intended to provide an overview or framework to understand the nature and character of the claims.

### BRIEF DESCRIPTION OF THE DRAWINGS

The accompanying drawings are included to provide a further understanding, and are incorporated in and constitute a part of this specification. The drawings illustrate one or more embodiment(s), and together with the Detailed Description explain the principles and operation of the various embodiments. As such, the disclosure will become more fully understood from the following Detailed Description, taken in conjunction with the accompanying Figures, in which:

FIG. 1A is an elevated view of an example DIOX glass substrate in the form of a planar substrate.

FIG. 1B is a close-up cross-sectional view of the DIOX substrate of FIG. 1A as taken in the x-y plane and that illustrates an example DIOX process that takes place across the substrate surface and into the body of the substrate.

FIG. 1C schematically illustrates the result of the DIOX process that forms the DIOX substrate, which has a near-surface spike region (R1) and a deep region (R2).

FIG. 2 is a representation of an example refractive index profile n(x) for the DIOX substrate with respect to the depth from the surface (illustrated in FIG. 1C), showing the spike region, the deep region, and the knee at the transition between the two regions.

FIG. 3A is a schematic diagram of an example prism-coupling system according to the disclosure and that is used to measure IOX articles using the methods disclosed herein.

FIG. 3B is a close-up view of the photodetector system of the prism-coupling system of FIG. 3A.

FIG. 3C is a schematic representation of a mode spectrum that includes TM and TE mode spectra as captured by the photodetector system of FIG. 3B.

FIG. 4A is a side view of an example light source having a light source device that supports multiple light source elements, wherein the light source device can be moved laterally by a linear actuator to align one or more light-emitting elements having a select wavelength with input optical axis.

FIG. 4B is a close-up side view of an example configuration of a light-emitting element and a narrow-band optical filter for a portion of the light source device of FIG. 4A.

FIG. 4C is a top-down view of an example light source device showing three light emitting elements arranged in a line and that emit different wavelengths of measurement light.

FIG. 4D is similar to FIG. 4C and illustrates an example light source device having pairs of light-emitting elements, wherein the different pairs emit different wavelengths of the measurement light.

FIGS. 4E and 4F are similar to FIG. 4A and show the light source device in two different lateral positions as established by the linear actuator, wherein the different lateral positions have a different light-emitting element and a different optical filter aligned with the input optical axis.

FIG. 5A is similar to FIG. 4A and illustrates an example configuration for the light source system wherein the light source device supports a single broadband light-emitting element and wherein the different optical filters are moved laterally by the linear actuator to align with the broadband light-emitting element to define sequential measurement light beams having different measurement wavelengths.

FIGS. 5B and 5C are top-down views of the support frame and the optical filters showing the lateral movement of the support frame and optical filters using guide features and the linear actuator.

FIG. 6A is similar to FIGS. 4A and 5A and illustrate an example of an optical filter system having a filter member that rotates a select optical filter into the input optical path formed by a broadband light-emitting element, different examples of which are shown in the two close-up insets.

FIG. 6B is a top-down view of an example round filter member having four different optical filters.

FIG. 6C is similar to FIG. 6B and illustrates an example of a non-round (eccentric) filter member having three different optical filters.

FIGS. 7A, 7B, and 7C are schematic diagrams that illustrate example configurations of the optical filter system as arranged on the detection side of the prism-coupling system.

FIG. 8 is a schematic diagram of a portion of an example mode spectrum similar to that of FIG. 3C and illustrating an example method of determining the fractional mode number from the measured mode spectrum for the TE and TM mode spectra.

FIG. 9 is a plot of the measured spike depth DOLₛₚ (μm) versus diffusion time t (hrs) for example 10× articles formed from a lithium-containing aluminosilicate glass substrate using a DIOX process, with the measurements performed by a single-wavelength prism-coupling system (open squares) and a three-wavelength prism-coupling system (dark circles).

FIG. 10 is a plot of the measured knee stress CSₖ (MPa) versus the TM fringe (mode) count N_{TM} based on the measurements made on example IOX articles formed from lithium-containing aluminosilicate glass substrates and using a same DIOX process where the diffusion time for the first diffusion step was the same but the diffusion time for the second diffusion step was varied for the different IOX articles, with the single-wavelength measurements shown by X's and the three-wavelength measurements shown by dark squares.

FIG. 11 is a plot of the measured knee stress CSₖ (MPa) after the two-step ion exchange (DIOX) versus ion-exchange time t (hours) for the same type of IOX articles as considered in FIG. 4.

FIG. 12 is a similar plot to that of FIG. 9 for additional example measurements.

FIG. 13A is a schematic diagram of an example TM and TE mode spectra pair for a single-wavelength measurement and that respectively include four TM and TE modes or fringes, and showing a measurement window size of 0.5 fringes.

FIG. 13B is similar to FIG. 8A except that it shows three TM and TE mode spectra pairs, one for each of three measurement wavelengths, and shows a larger effective measurement window of about 0.9 fringes, which is almost double that for the single-wavelength case of FIG. 8A.

FIG. 14A depicts a non-frangible test result on a test glass-based article in the form of an example IOX article.

FIG. 14B depicts a frangible test result on a test glass article in the form of an example IOX article.

### DETAILED DESCRIPTION

Reference is now made in detail to various embodiments of the disclosure, examples of which are illustrated in the accompanying drawings. Whenever possible, the same or like reference numbers and symbols are used throughout the drawings to refer to the same or like parts. The drawings are not necessarily to scale, and one skilled in the art will recognize where the drawings have been simplified to illustrate the key aspects of the disclosure.

The acronym IOX can mean either “ion exchange” or “ion exchanged,” depending on the context of the discussion. An “IOX article” means an article formed using at least one 10× process. Thus, an article formed by a DIOX process is referred to herein as an IOX article, though it could also be referred to as a DIOX article.

The term “glass based” is used herein to describe a material, article, matrix, substrate, etc., means that the material, article, matrix, material, substrate, etc. can comprise or consist of either a glass or a glass ceramic.

The compressive stress profile for an IOX article is denoted CS(x) and is also referred to herein as just the stress profile. The surface compressive stress or just “surface stress” for the stress profile is denoted CS and is the value of the compressive stress profile CS(x) for x=0, i.e., CS=CS(0), where x=0 corresponds to the surface of the IOX article.

The depth of compression DOC is the x distance into the IOX article as measured from the surface of the IOX article to where the compressive stress CS(x) or CS′(x) crosses zero.

The knee stress is denoted CSₖ and is the amount of compressive stress at a knee transition point (depth D1) between a spike region (R1) and a deep region (R2), i.e., CS(D1)=CSₖ.

The spike region R1 has a spike depth from the substrate surface that is denoted both as D1 and DOL_{SP}, with the latter also being referred to as the spike depth of layer. The spike region is also referred as a “near-surface spike region” to clarify the distinction with the deep region.

The deep region R2 has a depth D2 which is also denoted as the total depth of layer DOL_{T} for the total IOX region.

The acronym FWHM means “full-width half maximum.”

The terms “preferred measurement window” and “extended measurement window” are synonymous.

The abbreviation μm stands for micron or micrometer, which is 10⁻⁶ meter.

The abbreviation nm stands for nanometer, which is 10⁻⁹ meter.

The claims as set forth below are incorporated into and constitute part of this Detailed Description.

The term “contrast” as used herein with respect mode lines or fringes of a mode spectrum means a measure of the difference between a minimum intensity value and a maximum intensity value, and can include a rate of change in the intensity. One example measure of contrast C=(I_{MAX}−I_{MIN})/(I_{MAX}+I_{MIN}) where I_{MAX} and I_{MIN} are the maximum and minimum intensity values. Other measures of contrast used in the art of image processing can also be used.

Example prism-coupling systems and measurement methods are described for example in: U.S. Application Publication No. 2016/0356760, published Dec. 8, 2016, entitled “METHODS OF CHARACTERIZING ION-EXCHANGED CHEMICALLY STRENGTHENED GLASSES CONTAINING LITHIUM (also published as WO 2016/196748 A1); U.S. Pat. No. 9,897,574, issued Feb. 20, 2018, entitled “METHODS OF CHARACTERIZING ION-EXCHANGED CHEMICALLY STRENGTHENED GLASSES CONTAINING LITHIUM”; and U.S. Application Publication No. 2019/0033144, published Jan. 31, 2019, “METHODS OF IMPROVING THE MEASUREMENT OF KNEE STRESS IN ION-EXCHANGED CHEMICALLY STRENGTHENED GLASSES CONTAINING LITHIUM,” and U.S. Pat. No. 9,534,981, issued Jan. 3, 2017, “PRISM-COUPLING SYSTEMS AND METHODS FOR CHARACTERIZING ION-EXCHANGE WAVEGUIDES WITH LARGE DEPTH-OF-LAYER,” each of which is incorporated herein by reference in its entirety.

U.S. Pat. No. 10,732,059, issued Aug. 4, 2020, entitled “PRISM-COUPLING STRESS METER WITH WIDE METROLOGY PROCESS WINDOW,” is also incorporated herein by reference in its entirety.

IOX Article

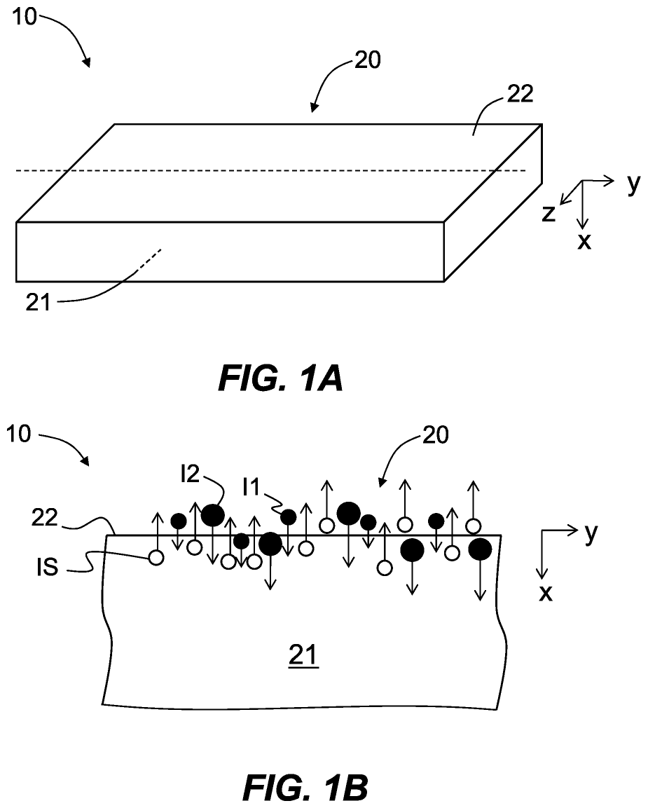

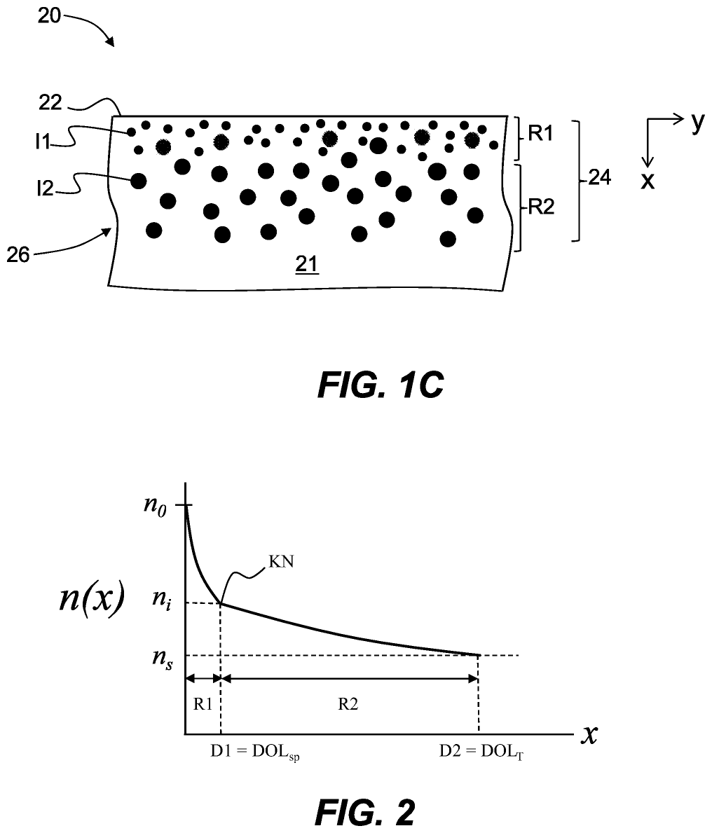

FIG. 1A is an elevated view of an example IOX article 10. The IOX article 10 comprises a glass-based substrate 20 having a matrix 21 that defines a (top) surface 22, wherein the matrix has a base (bulk) refractive index nₛ and a surface refractive index no. FIG. 1B is a close-up cross-sectional view of the IOX article 10 as taken in the x-y plane and illustrates an example DIOX process that takes place across the surface 22 and into the matrix 21 in the x-direction to form the example IOX article.

The substrate 20 includes in the matrix 21 substrate ions IS, which exchange for first ions I1 and second ions I2. The first and second ions I1 and I2 can be introduced into the matrix 21 either sequentially or concurrently using known techniques. For example, second ions I2 can be K⁺ ions introduced via a KNO₃ bath for strengthening, prior to introducing first ions I1 that can be Ag⁺ ions introduced via a AgNO₃-containing bath to add the anti-microbial property adjacent surface 22. The circles in FIG. 1B that represent ions I1 and I2 are used for schematic illustration only, and their relative sizes do not necessarily represent any actual relationship between the sizes of the actual ions participating in the ion exchange. FIG. 1C schematically illustrates the result of a DIOX process that forms the IOX article 10, wherein the substrate ions IS are omitted in FIG. 1C for ease of illustration and are understood as constituting the matrix 21. The DIOX process forms an IOX region 24 that includes a near-surface spike region R1 and a deep region R2, as explained below. The IOX region 24 defines an optical waveguide 26.

In addition, ions I1 may be present in significant numbers in both regions R1 and R2 (see FIG. 2, introduced and discussed below) as may be ions of type 12. Even with a one-step ion-exchange process it is possible to observe the formation of two IOX regions R1 and R2, with significant differences in the relative concentrations of ions I1 and I2. In an example, using an ion exchange of Na-containing or Li-containing glass in a bath containing a mixture of KNO₃ and AgNO₃, it is possible to obtain the spike region R1 with significant concentrations of both Ag⁺ and K⁺, and the deep region R2 also with significant concentrations of Ag⁺ and K⁺, but the relative concentration of Ag⁺ with respect to K⁺ may be significantly larger in the spike region R1 than in the deep region R2.

FIG. 2 is a representation of an example refractive index profile n(x) for an example IOX article 10, such as illustrated in FIG. 1C, and showing the spike region R1 associated with the shallower ion-exchange (ions I1) and that has a depth D1 (or DOLₛₚ) into the matrix 21. The deep region R2 associated with the deeper ion-exchange (ions I2) and has a depth D2 that defines the total depth-of-layer (DOL_{T}). In an example, the total DOL_{T} is at least 50 μm and further in an example can be as large as 150 μm or 200 μm. The transition between the spike region R1 and the deep region R2 defines a knee KN in the refractive index profile n(x) and also in the corresponding stress profile CS(x), as described below.

The deep region R2 may be produced in practice prior to the spike region R1. The spike region R1 is immediately adjacent the substrate surface 22 and is relatively steep and shallow (e.g., D1 is a few microns), whereas the deep region R2 is less steep and extends relatively deep into the substrate to the aforementioned depth D2. In an example, the spike region R1 has a maximum refractive index no at substrate surface 22 and steeply tapers off to an intermediate index nᵢ (which could also be called the “knee index”), while the deep region R2 tapers more gradually from the intermediate index down to the substrate (bulk) refractive index nₛ. It is emphasized here that other IOX processes can result in a steep and shallow near-surface refractive index change and that a DIOX process is discussed here by way of illustration.

In some examples, the IOX article 10 is frangible while in other examples, it is non-frangible, according to the frangibility criteria set forth below.

Prism-Coupling System

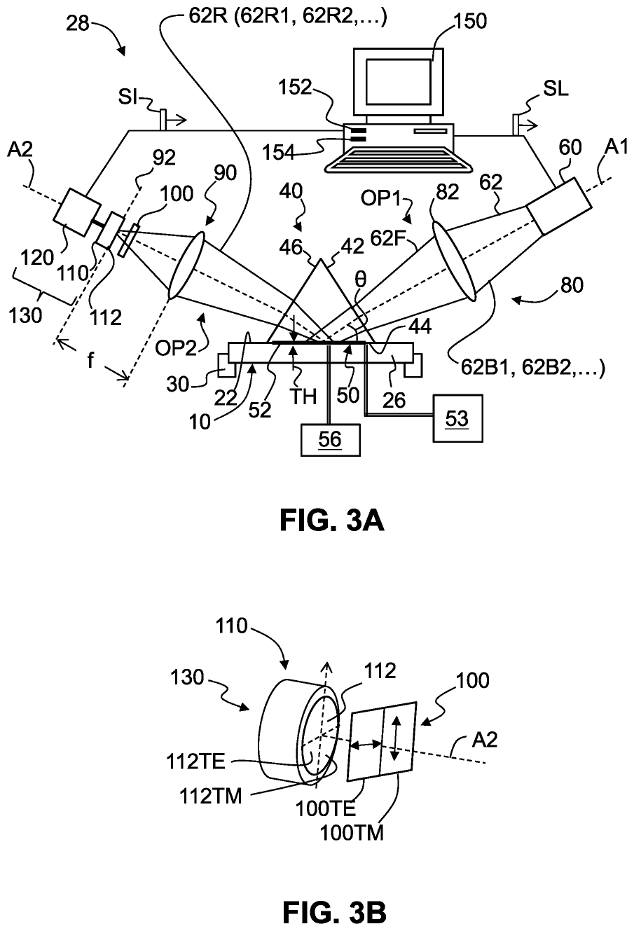

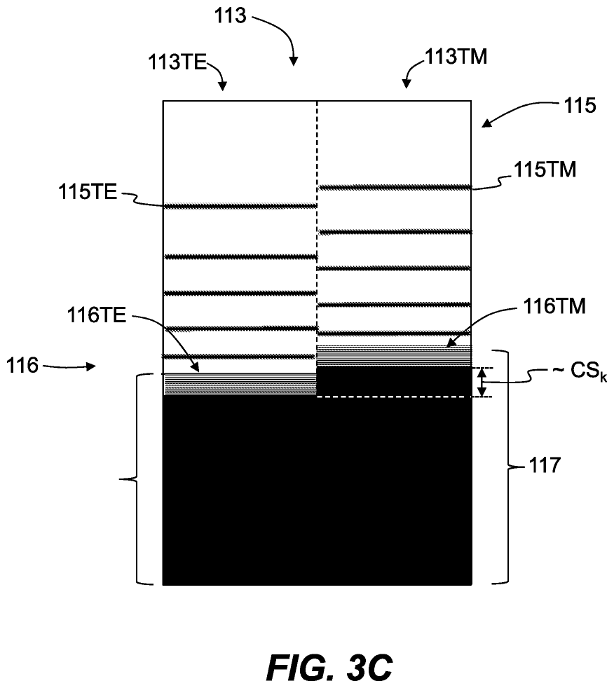

FIG. 3A is a schematic diagram of an example prism-coupling system 28 that can be used to carry out aspects of the methods disclosed herein. The prism coupling methods using the prism-coupling system 28 are non-destructive. This feature is particularly useful for measuring frangible IOX articles for research and development purposes and for quality control in manufacturing.

The prism-coupling system 28 includes a support stage 30 configured to operably support the IOX article 10. The prism-coupling system 28 also includes a coupling prism 40 that has an input surface 42, a coupling surface 44 and an output surface 46. The coupling prism 40 has a refractive index nₚ>ₙ. The coupling prism 40 is interfaced with the IOX article 10 being measured by bringing coupling-prism coupling surface 44 and the surface 22 into optical contact, thereby defining an interface 50 that in an example can include an interfacing (or index-matching) fluid 52 having a thickness TH. In an example, the prism-coupling system 28 includes an interfacing fluid supply 53 fluidly connected to the interface 50 to supply the interfacing fluid 52 to the interface. This configuration also allows for different interfacing fluids 52 with different refractive indices to be deployed. Thus, in an example, the refractive index of the interfacing fluid 52 can be changed by operation of the interfacing fluid supply 53 to add a higher-index or lower-index interfacing fluid. In an example, the interfacing fluid supply 53 is operably connected to and controlled by the controller 150.

In an exemplary measurement, a vacuum system 56 pneumatically connected to the interface 50 can be used to control the thickness TH by changing the amount of vacuum at the interface. In an example, the vacuum system is operably connected to and controlled by the controller 150.

The prism-coupling system 28 includes input and output optical axes A1 and A2 that respectively pass through the input and output surfaces 42 and 46 of the coupling prism 40 to generally converge at the interface 50 after accounting for refraction at the prism/air interfaces.

The prism-coupling system 28 includes, in order along the input optical axis A1, a light source system 60 that emits measurement light 62 in the general direction along the input optical axis A1. The measurement light 62 has a measurement wavelength λ, which can be sequentially changed during the operation of the prism-coupling system 28 to generate sequential input (measurement) light beams 62B1, 62B2, . . . having different measurement wavelengths λ. Example configurations of the light source system 60 that can be used to sequentially change the measurement wavelength λ are described in greater detail below. Note that the input optical axis A1 runs between the light source system 60 and the coupling prism 40. A focusing optical system 80 that includes a focusing lens 82 is used to focus the measurement light to form focused measurement light 62F.

The prism-coupling system 28 also includes, in order along the output optical axis A2 from the coupling prism 40, a collection optical system 90 having a focal plane 92 and a focal length f and that receives reflected light 62R as explained below, a TM/TE polarizer 100, and a photodetector system 130. In an example, the reflected light 62R comprises sequentially reflected light beams 62R1, 62R2, . . . each having a different measurement wavelength, as explained in greater detail below. The portion of the prism-coupling system 28 downstream of the coupling prism 40 (as defined by the direction of travel of the measurement light 62) is referred to as the detector side of the system.

The input optical axis A1 defines the center of an input optical path OP1 between the light source system 60 and the coupling surface 44. The input optical axis A1 also defines a coupling angle θ with respect to the surface 22 of the IOX article 10 being measured.

The output optical axis A2 defines the center of an output optical path OP2 between the coupling surface 44 and the photodetector system 130. Note that the input and output optical axes A1 and A2 may be bent at the input and output surfaces 42 and 46, respectively, due to refraction. They may also be broken into sub-paths by inserting mirrors (not shown) into the input and output optical paths OP1 and/or OP2.

In an example, the photodetector system 130 includes a detector (camera) 110 and a frame grabber 120. In other embodiments discussed below, the photodetector system 130 includes a CMOS or CCD camera. FIG. 3B is a close-up elevated view of the TM/TE polarizer 100 and the detector 110 of the photodetector system 130. In an example, the TM/TE polarizer includes a TM section 100TM and a TE section 100TE. The photodetector system 130 includes a photosensitive surface 112.

The photosensitive surface 112 resides in the focal plane 92 of the collecting optical system 90, with the photosensitive surface being generally perpendicular to the output optical axis A2. This serves to convert the angular distribution of the reflected light 62R exiting the coupling prism output surface 46 to a transverse spatial distribution of light at the sensor plane of the detector 110. In an example embodiment, the photosensitive surface 112 comprises pixels, i.e., the detector 110 is a digital detector, e.g., a digital camera.

Splitting the photosensitive surface 112 into TE and TM sections 112TE and 112TM as shown in FIG. 3B allows for the simultaneous recording of digital images of the angular reflection spectrum (mode spectrum) 113, which includes the individual TE and TM mode spectra 113TE and 113TM for the TE and TM polarizations of the reflected light 62R. This simultaneous detection eliminates a source of measurement noise that could arise from making the TE and TM measurements at different times, given that system parameters can drift with time.

FIG. 3C is a schematic representation of a mode spectrum 113 as captured by the photodetector system 130. The mode spectrum 113 has total-internal-reflection (TIR) section 115 associated with guided modes and a non-TIR section 117 associated with radiation modes and leaky modes. A transition 116 between the TIR section 115 and the non-TIR section 117 defines a critical angle and is referred to as the critical angle transition 116, and is denoted 116TM for the TM mode spectrum 113TM and 116TE for the TE mode spectrum. The difference in locations of the start of the critical angle transitions 116TM and 116TE for the TM and TE mode spectra 113TM and 113TE is proportional to the knee stress CSₖ and this is proportionality is indicated by “^{˜}CSₖ” in FIG. 3C.

The TM mode spectrum 113TM includes mode lines or fringes 115TM while the TE mode spectrum 113TE includes mode lines or fringes 115TE. The mode lines or fringes 115TM and 115TE can either be bright lines or dark lines, depending on the configuration of the prism-coupling system 28. In FIG. 3C, the mode lines or fringes 115TM and 115TE are shown as dark lines for ease of illustration. In the discussion below, the term “fringes” is used as short-hand for the more formal term “mode lines.”

The stress characteristics are calculated based on the difference in positions of the TM and TE fringes 115TM and 115TE in the mode spectrum 113. At least two fringes 115TM for the TM mode spectrum 113TM and at least two fringes 115TE for the TE mode spectrum 113TE are needed to calculate the surface stress CS. Additional fringes are needed to calculate the stress profile CS(x). The TM and TE fringes 115TM and 115TE also need to have a suitable contrast so that their positions can be accurately determined.

With reference again to FIG. 3A, the prism-coupling system 28 includes a controller 150, which is configured to control the operation of the prism-coupling system. The controller 150 is also configured to receive and process from the photodetector system 130 image signals SI representative of captured (detected) TE and TM mode spectra images. The controller 150 includes a processor 152 and a memory unit (“memory”) 154. The controller 150 may control the activation and operation of the light source system 60 via a light-source control signal SL, and receives and processes image signals SI from the photodetector system 130 (e.g., from the frame grabber 120, as shown). The controller 150 is programmable (e.g., with instructions embodied in a non-transitory computer-readable medium) to perform the functions described herein, including the operation of the prism-coupling system 28 and the aforementioned signal processing of the image signals SI to arrive at a measurement of one or more of the aforementioned stress characteristics of the IOX article 10.

Example Light Source Systems

A. Translatable Light Source Device

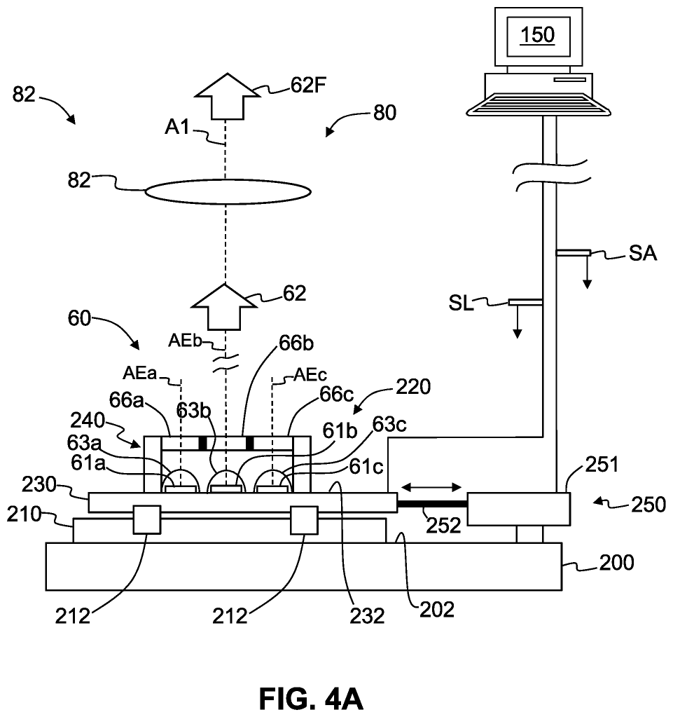

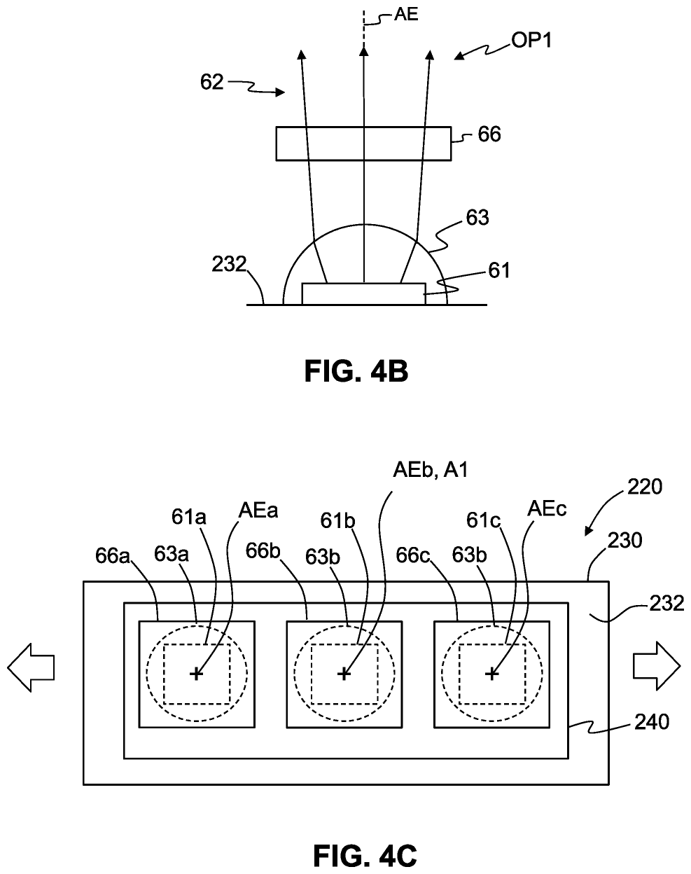

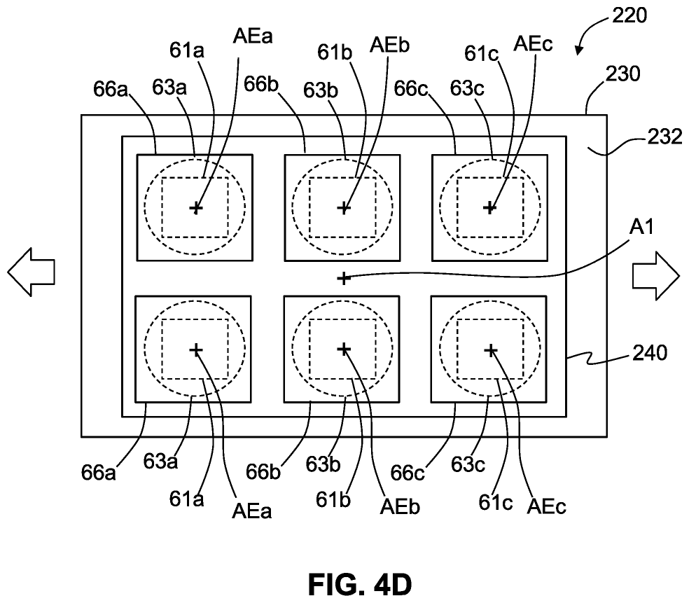

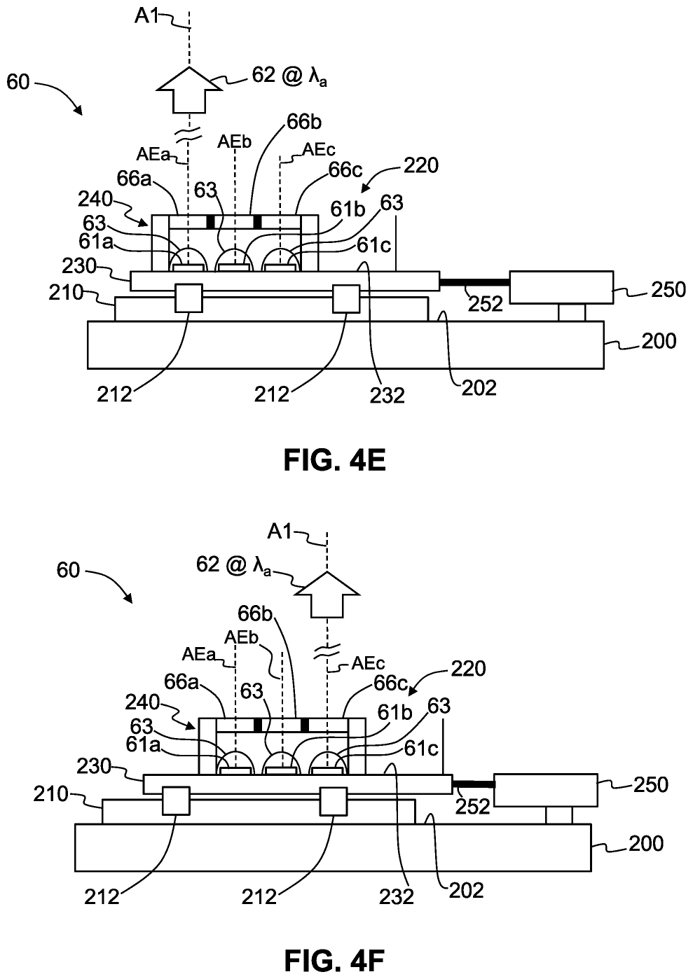

FIG. 4A is a schematic diagram of a first example light source system 60. The light source system 60 includes a support base 200 having a top surface 202 that supports a guide rail 210. The guide rail 210 movably supports guide-rail mounts 212, which in an example slide along the guide rail. The guide-rail mounts 212 operably support a light source device 220. The light source device 220 includes a support substrate 230 having a top surface 232. The support substrate 230 can include electrical wiring, circuitry and other electronic components (not shown). In an example, the support substrate 230 can comprise a printed circuit board (PCB). The support substrate 230 supports on its top surface 232 a plurality of light-emitting elements 61, with an example light-emitting element and associated components shown in the close-up side view of FIG. 4B.

Three example light-emitting elements 61 are shown in FIG. 4A and are denoted 61a, 61b and 61c and each emits measurement light 62 having a different measurement wavelength λ, e.g., λₐ, λ_{b} and λ_{c}, respectively. In an example, the light-emitting elements 61 comprise light-emitting diodes (LEDs) or laser diodes. Three example measurement wavelengths λₐ, λ_{b} and λ_{c} can respectively include 540 nm, 595 nm and 650 nm. In an example, the measurement wavelengths λ fall within the wavelength range from 350 nm to 850 nm, or in the more narrow wavelength range from 540 nm to 650 nm. In an example, the measurement wavelength is a center wavelength of a relatively narrow wavelength band. FIG. 4A shows the example focusing lens 82 of the focusing optical system 80, which is used to receive the measurement light 62 and form focused measurement light 62F.

In the example shown, each light-emitting element 61 is encapsulated within a translucent case 63 (e.g., cases 63a, 63b and 63c) that in an example can act as a lens. Each light-emitting element 61 has a central axis AE, with the axes for light-emitting elements 61a, 61b and 61c respectively denoted as AEa, AEb and AEc. Note that three light-emitting elements 61 are shown by way of example and that fewer (i.e., two) light-emitting elements 61 can be used or more than three light-emitting elements can be used.

The light source system 60 also can include an array of two or more optical filters 66 respectively operably disposed adjacent the light-emitting elements 61. FIG. 4A shows three optical filters 66a, 66b and 66c respectively operably disposed adjacent light-emitting elements 61a, 61b and 61c along the respective axes AEa, AEb and AEc. In an example, the optical filters 66 are supported by a support frame 240 attached to the top surface 232 of the support substrate. Each optical filter 66a, 66b and 66c has a relatively narrow band pass centered on the wavelength λₐ, λ_{b} and λ_{c}, respectively. The wavelength band pass of the optical filters 66 is narrower than the wavelength bandwidth of the corresponding light-emitting elements 61. Having a narrow wavelength band for the measurement light 62 allows for sharper TM and TE fringes 115TM and 115TE, respectively (see FIG. 3C).

FIG. 4C is a top-down view of an example light source device 220 showing the three light-emitting elements 61a, 61b and 61c arranged in a line. In this configuration where each light-emitting element 61 emits a unique measurement wavelength λ, a given light-emitting element (e.g., light-emitting element 61b, as shown) can be centered on the input optical axis A1, i.e., the central axis AE of the given light-emitting element can be co-axial with the input optical axis A1.

FIG. 4D is similar to FIG. 4C and shows an example configuration of the light source device 220 having a pair of light-emitting elements 61a, a pair of light-emitting elements 61b and a pair of light-emitting elements 61c, with these pair of light-emitting elements aligned with corresponding pairs of optical filters 66a, 66b and 66c. In this configuration, a given pair of the light-emitting elements 61 can be centered around the input optical axis A1, which is shown in FIG. 4D as residing between the pair of light-emitting elements 61b by way of example. Other configurations for the light source device 220 are also contemplated, such as arrangements of three or more light-emitting elements 61 respectively having a triangular arrangement, square arrangement, etc.

With reference again to FIG. 4A, the light source device 220 is mechanically connected to a motion control system 250. In the example shown, the motion control system comprises a linear actuator 251 and a drive shaft 252. Other example motion control systems 250 can be employed as known in the art. Parts of the discussion below refer to the linear actuator 251 and drive shaft 252 by way of example and for ease of discussion.

The motion control system 250 can be electrically connected to the controller 150, which can control the linear actuator via an actuator control signal SA to move the light source device 220 back and forth relative to the input optical axis A1 (e.g., in a direction perpendicular thereto). This lateral movement can be used to position (translate) a select one of the light-emitting elements 61a, 61b or 61c to be co-axial with or otherwise aligned with the input optical axis A1, as illustrated in the examples of FIGS. 4C and 4D.

FIGS. 4E and 4F are similar to FIG. 4A and show the light source device 220 in two different lateral positions as established by the linear actuator 251, wherein the different lateral positions have a different light-emitting element 61 and its corresponding optical filter 66 aligned with the input optical axis A1.

When the light source 60 in FIG. 4A sequentially generates measurement light beams 62B1, 62B2, . . . having different wavelengths, the reflected light 62 comprises sequentially reflected light beams 62R1, 62R2, . . . each having a different wavelength (e.g., center wavelength). These sequentially reflected light beams are collected by the collection optical system 90 and detected at the detector 110 to digitally capture TM and TE mode spectra 113—one for each of the sequentially reflected light beams and thus for each of the measurement wavelengths (see FIG. 3A).

B. Broadband Light-Emitting Element with Translatable Filters

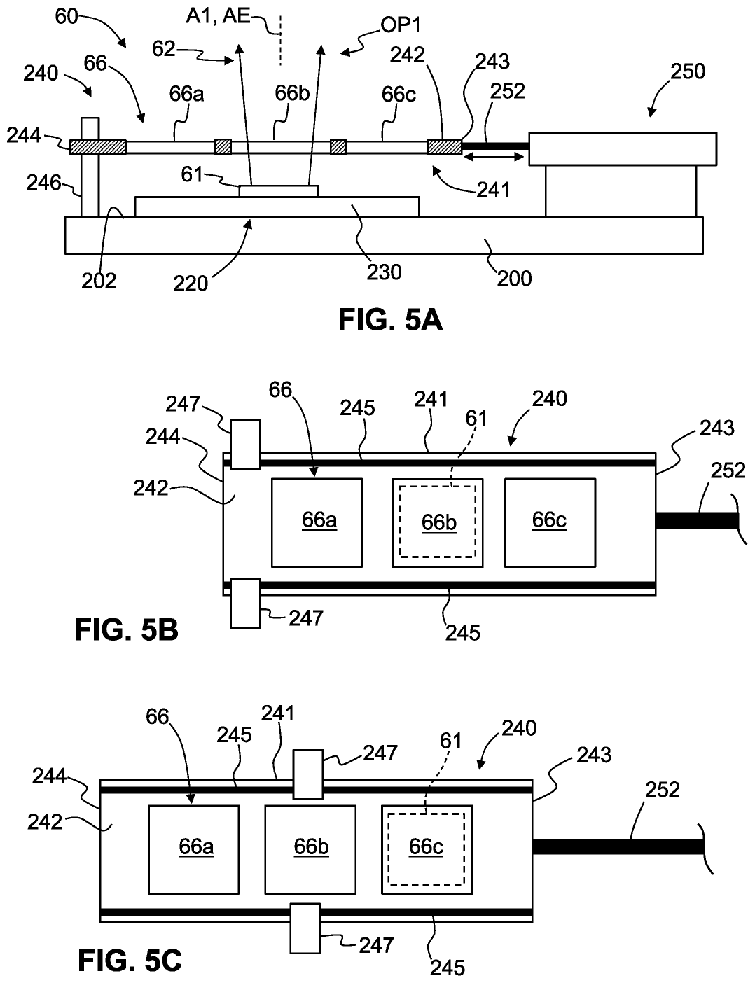

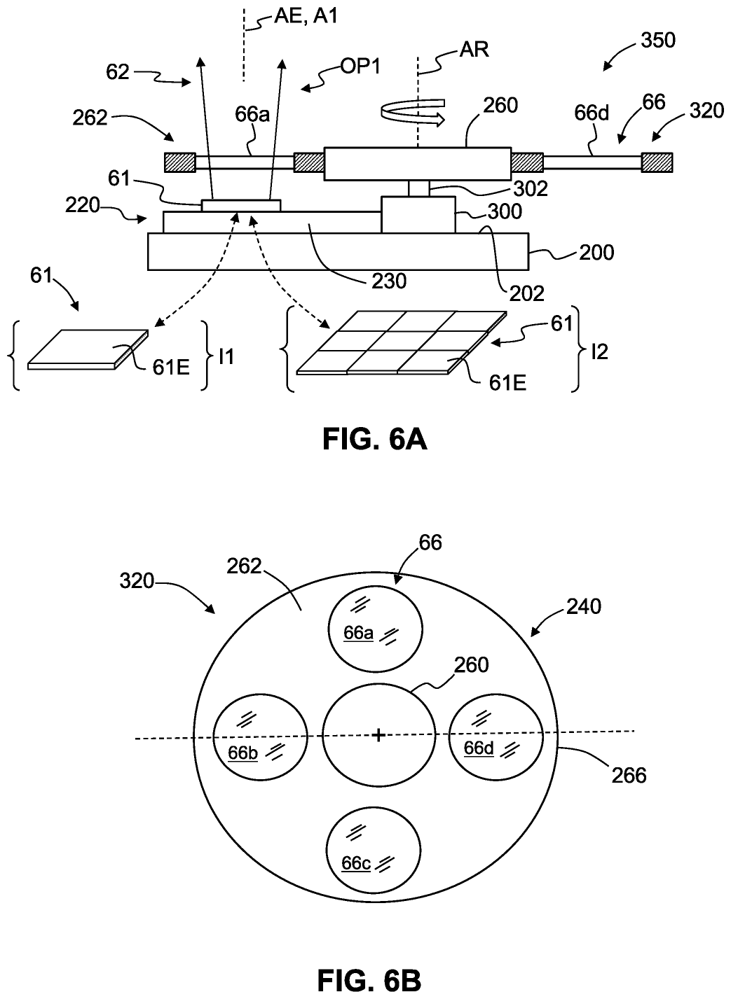

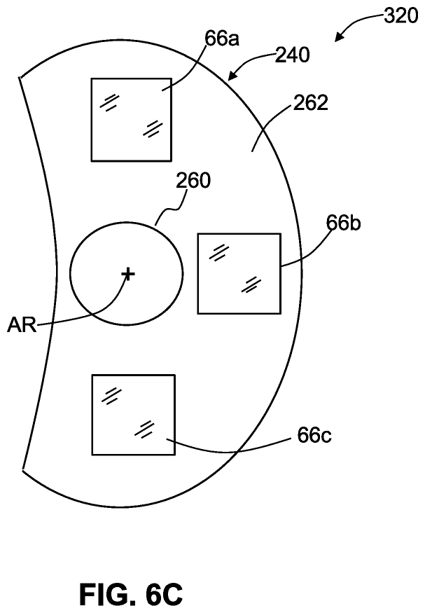

FIG. 5A is similar to FIG. 4A and shows an example of the light source system 60 wherein the light source device 220 has a single broadband light-emitting element 61 with a central axis AE co-axial with the input optical axis A1. A plurality of optical filters 66 (e.g., 66a, 66b, 66c, . . . ) are supported in a top section 241 of the support frame 240. FIG. 5B is a top-down view of the optical filters 66a, 66b and 66b supported in the top section 241 of the support frame 240. The light-emitting element 61 is shown in phantom as residing directly beneath the central optical filter 66b. In an example, multiple broadband light-emitting elements 61 can also be used, depending on how much intensity in the measurement light 62 is desired. The multiple broadband light-emitting elements 61 can be arranged tightly about the input optical axis A1 so that they collectively operate as a single large broadband on-axis light emitter. The single broadband light-emitting element 61 shown in the Figures is schematic and in an example is representative of multiple, tightly arranged broadband light emitters (see, e.g., FIG. 6A, discussed below).

The top section 241 of the support frame 240 has a top surface 242, a proximal end 243 and a distal end 244. The top surface 242 includes at least one guide feature 245, such as a pair of guide rails or guide grooves, as best seen in FIG. 5B. The support frame 240 also includes a support wall 246 that includes at least one guide feature 247 that is complementary to the at least one guide feature 245 of the top surface of the top section so that the top section can be slidably engaged by the support wall and move laterally in a guided manner.

The proximal end 243 of the top section 241 is operably engaged by the motion control system 250, e.g., by the drive shaft 252 of the linear actuator 251, to drive the lateral movement of the top section 241. FIG. 5C is similar to FIG. 5B and shows the top section 241 shifted to the left so that now the light-emitting element 61 resides directly beneath the optical filter 66c. Thus, the motion control system 250 can be used to control (e.g., via the controller 15) the lateral movement of the top section 241 to position a select one of the filters 66 to be in line with the light-emitting element 61 to define a select wavelength λₐ, λ_{b}, λ_{c}, etc. for the measurement light 62. The lateral position of the top section 241 is readily tracked the motion control system 250 and/or by the controller 150 so that a given optical filter 66 can be accurately aligned with the light-emitting element 61.

C. Light-Emitting Element with Rotatable Filters

FIG. 6A is similar to FIG. 5A and illustrates an example of the light source system 60 wherein the optical filters 66 are supported in a rotatable support frame 240. FIG. 6B is a top down view of an example rotatable support frame 240. The support frame 240 includes a central section 260 with a rotation axis AR, and an outer section 262 that supports multiple optical filters 66, e.g., 66a through 66d as shown by way of example. In an example, the outer section 262 has a perimeter 266 and is annular and the optical filters 66 are evenly distributed over the annular outer section. FIG. 6C is similar to FIG. 6B and illustrates another embodiment of the support frame 240 having a non-circular or eccentric shape. The close-up insets I1 and I2 of FIG. 6A show examples of the light-emitting element 61 comprising only single light emitter 61E and comprising multiple light emitters 61E.

The light source system 60 includes a drive motor 300 having a drive shaft 302 that is operably attached to the central section 260 so that the support frame 240 can be rotated about the rotation axis AR. In an example, the drive motor 300 is configured to rotate the support frame 240 in steps (e.g., angular increments) to position a select one of the optical filters 66 so that it resides just above the broadband light-emitting element 61, i.e., in the input optical path OP1. In an example, the drive motor 300 is connected to and controlled by the controller 150. The support frame 240 and optical filters 66 supported thereby constitute a filter member 320. In an example, the filter member comprises a filter wheel. The filter member 320, the drive motor 300 and the drive shaft 302 operably connected to the filter member at the central section 260 of the support frame 240 constitute an optical filter system 350.

B. Optical Filter System in the Collection Optical System

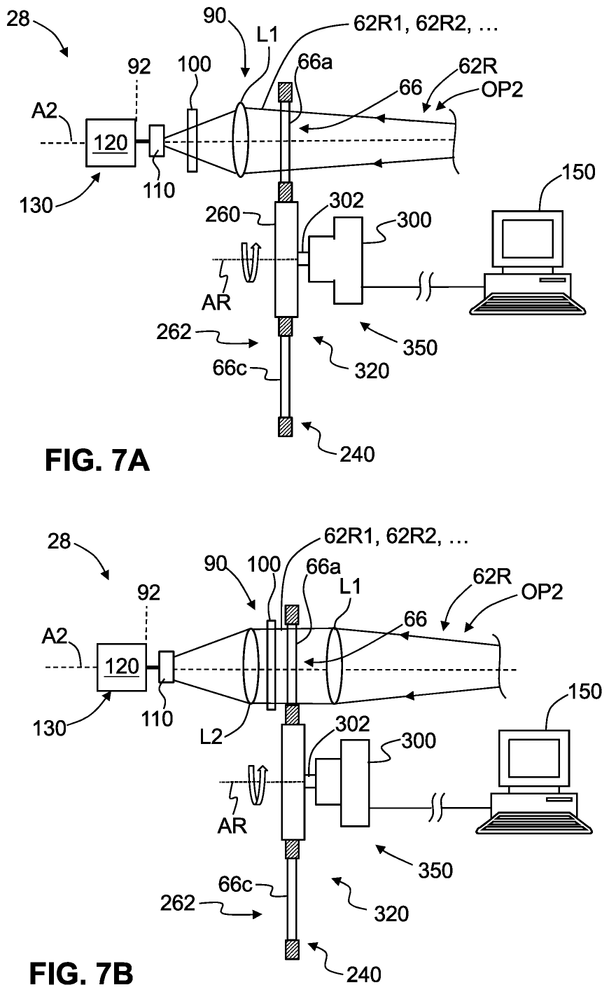

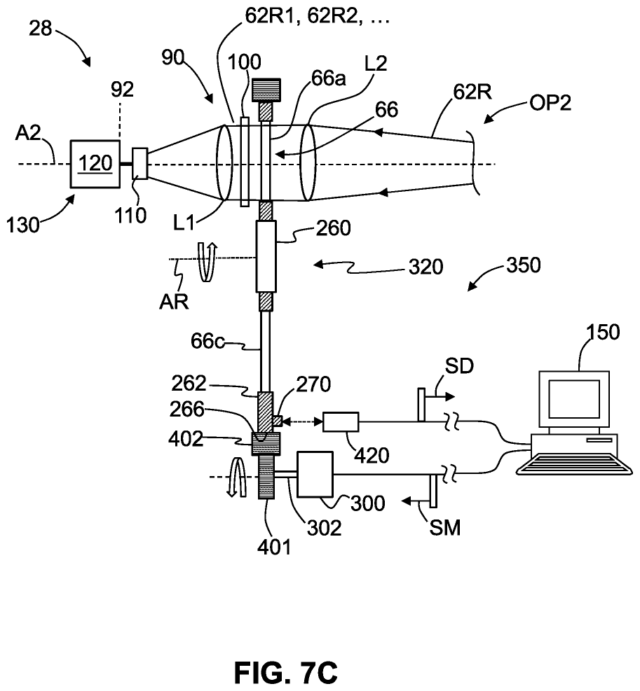

FIG. 7A is a schematic diagram that illustrates an example configuration of the prism-coupling system 28 wherein the optical filter system 350 is arranged on the detection side of the prism-coupling system 28 (see FIG. 3A) rather than on the light-source side. The optical filter system 350 is arranged so that the optical filters 66 can be operably disposed in the output coupling path OP2 to wavelength filter the reflected light 62R to form sequential narrow-band reflected light beams 62R1, 62R2, . . . .

In the example of FIG. 7A, the optical filter system 350 can reside in the output coupling path OP2 anywhere between the collection optical system 90 and the coupling prism 40. In some examples, it may be advantageous to place the optical filter system proximate the collection optical system 90, such as adjacent a collection lens L1 of the collection optical system as shown.

FIG. 7B is similar to FIG. 7A and illustrates an example wherein the optical filter system 350 is arranged so that the filter member 320 at least partially resides within the collection optical system 90, e.g., between first and second lenses L1 and L2 of the collection optical system. In this configuration, the reflected light 62R is substantially collimated by the first lens L1. This allows for the reflected light 62R to pass through the given optical filter 66 (e.g., 66a, as shown) at substantially normal incidence when forming the sequential narrow-band reflected light beams 62R1, 62R2, . . . .

In an example, the TM/TE polarizer 100 can also be positioned within the collection optical system 90 so that the substantially collimated reflected light 62R can also pass through the TM/TE polarizer at substantially normal incidence. The second lens L2 can serve as a focusing lens that directs the wavelength-filtered reflected measurement light (i.e., the sequential narrow-band reflected light beams 62R1, 62R2, . . . ) to the detector 110.

FIG. 7C is similar to FIG. 7B and illustrates an example wherein the drive shaft 302 of the drive motor 300 is connected to a first gear 401. The first gear 401 operably engages a second gear 402 that runs around at least a portion of the perimeter 266 of the outer section 262 of the support frame 240. The rotation of the drive shaft 320 rotates the first gear 401, which in turn rotates the second gear 402, which in turn rotates the filter member 302. As in the other embodiments, the filter member 320 is rotated to place a select filter 66 in the output coupling path OP2 to filter the reflected light 62R and form the sequential narrow-band reflected light beams 62R1, 62R2, etc.

In an example, a reference feature 270 is included on the guide member 320. The position of the reference feature 270 can be detected by a detection system 420. In an example, the reference feature 270 can be a protrusion or recess and the detection system 420 can be a distance sensor that senses a distance to the filter member 320, and wherein the distance is changed by the protrusion or recess. In another example, the reference feature 270 can be a reflective element, a bar code or like indicia and the detection system 420 can be scanner or machine vision system, etc. The detection system 420 and the drive motor 300 can be operably connected to the controller 150. The detection system 420 can provide to the controller 150 a detection signal SD representative of a rotational position of the filter member 320 and thus the relative positions of the filters 66 relative to the output optical path OP2. The controller 150 can also send a motor control signal SM to the drive motor 300 to cause the drive motor to place the filter member 320 in a select rotational position, e.g., with a select one of the filters 66 in the output coupling path OP2.

Measuring the IOX Article Using Different Measurement Wavelengths

A proper measurement of a stress characteristic of the IOX article 10 conventionally requires that the prism-coupling system 28 couple the focused measurement light 62F from the light source 60 (by focusing optical system 80) into a sufficient number of the guided modes supported by the IOX waveguide 26 so that most if not all of the refractive index profile in the spike region R1 as well as the deep region R2 is sampled so that the measured mode spectrum 113 is complete and accurate, i.e., includes information about the entire IOX region 24 and not just a one part of the IOX region.

When a guided or leaky mode associated with the spike region R1 has an effective index that is close to the critical angle, determining the precise location of the critical angle transition 116 in the mode spectrum 113 can be problematic. This is because the usual location of the maximum slope in the intensity profile can correspond to a slightly different effective index than the actual effective index at the spike depth D1, i.e., at the knee KN formed by the transition between the spike region and the deep region R2 (see FIG. 2).

As noted above, the resonance caused by the nearby guided or leaky mode in the effective-index spectrum can cause a significant change in the shape of the intensity distribution in the vicinity of the effective index corresponding to the index at the knee KN. Also as noted above, this can substantially distort the calculation of the fractional number of TE and TM fringes 115TE and 115TM, and hence of the spike depth D1, and thus the calculation of the knee stress CSₖ. This is particularly true for Li-based glass substrates 20 that undergo a DIOX process using Na⁺ and K⁺ ions to form the IOX article 10.

The above-described calculation distortions impose severe restrictions when using prism-coupling measurements for quality control of IOX articles 10 since an accurate estimation of the knee stress CSₖ is only possible in a narrow range of conditions (i.e., a narrow measurement process window) where the critical-angle intensity transitions 116 (see FIG. 3C) is unperturbed for both the TM and TE polarizations.

The systems and methods disclosed herein allow for making measurements of the IOX article 10 using different measurement wavelengths λ to obtain TM and TE mode spectra 113TM and 113TE having suitable contrast for performing an accurate measurement of a stress characteristic of the IOX article. This includes sequentially changing the measurement wavelength λ so that different measurement wavelengths can be sequentially coupled into the waveguide 26 of the IOX article 10 to obtain TM and TE mode spectra 113TM and 113TE for a preferred measurement window.

In a first step of the method, the IOX article 10 is loaded into the prism-coupling system 28 and a first mode spectrum 113 is collected as described above for a first measurement wavelength λ.

In a second step of the method, the first TM and TE spectra 113TM and 113TE are processed to obtain a TM and TE signal of intensity versus position of the respective fringes 115TM and 115TE captured by the photosensitive surface 112 of the photodetector system 130. This is equivalent to the intensity vs. coupling angle θ, which is also equivalent to the intensity vs. effective index n_{eff}, as there is a one-to-one relationship between position on the photosensitive surface 112, the coupling angle θ, and the effective index n_{eff} of guided optical modes propagating in the waveguide 26 defined by the IOX region 24 in the IOX article 10.

In a third step, the intensity versus position data from the second step is used to establish whether the first TM and TE mode spectra 113TM and 113TE were obtained (or reside in) a preferred measurement window of the prism-coupling system 28. In one example, this includes determining the fractional part of the full (real-number) mode count or fringe count for the TM mode spectrum 113TM and the TE mode spectrum 113TE. The full mode count includes an integer portion equal to the number of guided modes for the specific polarization (TM or TE), which is the same as the number of fringes 115TM or 115TE occurring in the TIR section 117 of the respective mode spectra 113TM or 113TE at the measurement wavelength. The number of TM fringes 115TM is N_{TM} while the number of TE fringes 115TE is N_{TE}.

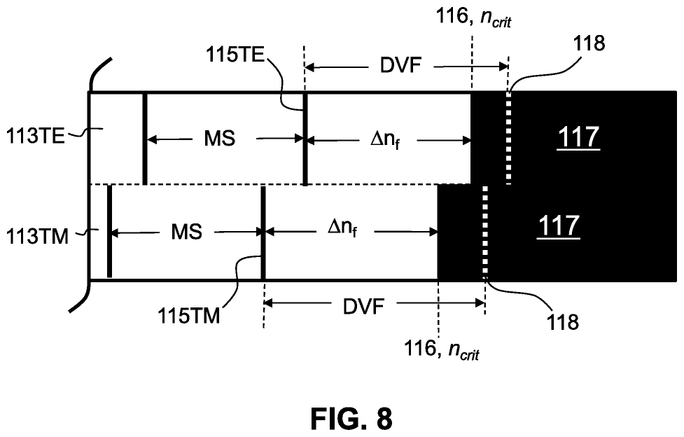

An aspect of the methods disclosed herein involves determining a fractional part FP of the number of modes (mode number) for both the TE mode spectrum 113TE and the TM mode spectrum 113TM. FIG. 8 is a schematic diagram of a portion of an example mode spectrum 113 similar to FIG. 3C and illustrates how the fractional part FP of the mode number for the TE mode spectrum 113TE and the TM mode spectrum 113TM can be determined.

In an example, the fractional part FP of the mode number is determined by comparing the distance between the last guided mode having the lowest effective index n_{eff}, and the effective index n_{eff} corresponding to the critical angle transition 116. For coupling angles θ beyond the critical angle, only part of the incident light 62F is reflected to form reflected light 62R, with the non-reflected portion of the incident light penetrating the IOX article 10 substantially deeper than the spike depth D1 as a leaky mode or a radiation mode.

The effective index n_{eff} corresponding to the critical angle is referred to as the “critical index” and is denoted n_{crit}. In some cases, the critical index n_{crit} can equal the substrate refractive index nₛ. For example, this situation can arise when the IOX article 10 is formed from an Li-containing glass substrate 20 that is chemically strengthened in a bath containing Na⁺ (e.g., NaNO₃).

The distance between the last guided mode and the critical angle n_{crit} corresponds to the difference Δn_{f} in effective index between the index of the last guided mode and the critical index is given by:

Δn_{f}=min(n_{eff})−n_{crit}

where min(n_{eff}) is the smallest of the effective indices of all guided modes for the specific polarization (TM or TE), and n_{crit} is the critical index for the same polarization.

The fractional part FP of the mode count (i.e., number of fringes) N_{TM} or N_{TE} is found by examining the space between the last guided mode 115TE or 115TM and the critical index n_{crit}. In some embodiments, the fractional part of the TM or TE mode count is determined by comparing Δn_{f} to the expected spacing to the next mode by extrapolating the dependence of effective index on the mode count. In some embodiments, a fit of the dependence of effective index n_{eff} on the mode count can be obtained from the integer-numbered guided modes. The fit is then extrapolated, and a mode number is assigned to the critical angle from the value of the mode count N_{TM} or N_{TE} at which the extrapolated function equates to the measured n_{crit}. This same procedure may be performed directly using the position of fringes 115TM or 115TE in the given mode spectrum 113TM or 113TE versus the fringe number, or an angle in the angular spectrum versus fringe number.

With continuing reference to FIG. 8, one method of determining the fractional part FP of the fringe count is to consider a virtual fringe 118 that would be the next fringe in the given mode spectrum but for the fact that the critical angle transition 116 cuts off the virtual fringe. This can be accomplished by an extrapolation based the existing fringe spacings. The distance from the last fringe 115TE or 115TM to the corresponding virtual fringe 118 is DVF, so that the fractional part FP of the mode (fringe) count is FP=Δn_{f}/DVF, noting that Δn_{f} and DVF can be different for the TM mode spectrum 113TM and the TE mode spectrum 113TE.

Another method of determining the fractional part FP of the fringe count is when there are only two or three modes. In this case, one can approximate the distance DVF by the spacing MS between the two modes closest to the TIR-PIR transition, as also shown in FIG. 3E.

In one example, to be within the preferred measurement window, the fractional part FP of the fringe count N_{TM} or N_{TE} is within a select range. In one example, the range on the fraction portion FP of the fringe count is 0.1 to 0.85. In another example, the fractional part FP of the fringe count can be greater than 0.15. In another example, the fractional part FP of the fringe count can be below 0.8, e.g., smaller than 0.75, or smaller than 0.70. Thus, example ranges on FP include 0.15 and 0.75 or 0.15 and 0.70.

In an example, TM and TE mode spectra 113TM and 113TE having fractional parts FP that fall within at least one of the example FP ranges set forth above can be taken as a “best” TM and TE mode spectra from a set of TM and TE mode spectra taken at different wavelengths. In another example, the TM and TE mode spectra 113TM and 113TE having the greatest fringe contrast is taken as the “best” TM and TE mode spectra from a set of TM and TE mode spectra taken at different wavelengths. In an example, the best TM and TE mode spectra from a set of TM and TE mode spectra taken at different wavelengths has the greatest fringe contrast and has an fractional part within one of the FP ranges cited above. If multiple pairs of TM and TE mode spectra 113TM and 113TE fall within a select FP ranges, then in an example the TM and TE mode spectra whose modes (fringes) are least affected (i.e., least distorted) by the critical angle transitions 116TM and 116TE is selected. Various selection criteria for what constitutes when a mode (fringe) is least affected by the corresponding critical transition are discussed below.

If the fractional part FP of at least one of the TM mode spectrum 113M and the TE mode spectrum 113TE is outside of the select range, then the prism-coupling system 28 is set to a different measurement condition that brings the fraction portion FP of the fringe count to within the select range, which in turn allows for determining the at least one stress parameter of the IOX article 10 with better accuracy.

In another example, to be within the preferred measurement window, there can be no guided or a leaky mode close enough to the critical index n_{crit} to substantially alter the shape (intensity profile) of the critical angle transition 116. This is because location of the maximum intensity slope of the critical angle transition 116 is used to determine the stress-related parameters of the IOX article 10. A guided or leaky mode resonance that adversely affects the critical-angle intensity transition in the captured prism-coupling spectrum is referred to herein as an offending resonance or an offending mode.

As utilized herein, an optical propagation mode is referred to as “guided” or “bound” if its effective index is higher than the critical index. As utilized herein, an optical propagation mode is referred to as “leaky” if its effective index is lower than the critical index. A leaky mode produces a transmission resonance when its effective index is relatively close to the critical index, particularly if it is substantially closer to the mode spacing of the last two guided modes, i.e., the two guided modes with the lowest effective index for a particular polarization.

As utilized herein, the “transmission resonance” refers to a dip in the intensity in a given mode spectrum 113TM or 113TE where the intensity would normally monotonically decrease with decreasing effective index for n_{eff}<n_{crit}. When the dip in the mode spectrum gets very close to the critical-angle transition 116, the location of maximum slope shifts toward a slightly larger effective index, which corresponds to the lowest material index near the bottom of the spike region R1.

In a similar way, a guided mode with an effective index only slightly above the critical index may cause the intensity in the vicinity of the critical angle transition 116 to change due to the nonzero breadth of the coupling resonance for the mode. The nonzero breadth may be a result of several factors, including the coupling strength, the resolution of the optical system in the prism-coupling system 28, and aberrations caused by warp of the IOX article 10 in the measurement area.

In each of the above cases, the apparent location of the critical angle in the measured mode spectrum 113TM or 113TE is shifted significantly when the location of the corresponding resonance (bound-mode or leaky-mode resonance) is within a distance from the critical angle that is about the same as the breadth of the resonance in terms of effective index, or smaller.

Hence, a measured mode spectrum 113TM or 113TE may be considered outside of the preferred measurement window when a guided mode is within 0.5 FWHM, such as within 0.6 FWHM or 0.7 FWHM of the breadth of the guided-mode resonance. Similarly, a measured mode spectrum 113TM or 113TE is considered outside of the preferred measurement window when the lowest-intensity point of a leaky mode is within 0.5 FWHM, such as within 0.6 FWHM or 0.7 FWHM breadth of the leaky-mode resonance.

When the leaky-mode resonance is somewhat farther away from the critical index n_{crit}, the resonance is broad and asymmetric, and its FWHM may be challenging to measure and define in industrial measurement conditions. Hence, in some embodiments, a different criterion can be used to identify whether a given leaky mode adversely affects the critical angle transition 116. In one such method, the distance between the lowest-intensity point (the dip location) of the leaky mode and the apparent position of the critical angle transition 116 is considered.

The measured mode spectrum 113TM or 113TE may be considered within the preferred measurement window when the distance between the leaky mode dip location and the apparent location of the transition is smaller than 0.2 times the distance from the apparent critical angle transition to the nearest guided-mode location, or smaller than 0.3, 0.4, or 0.5 times the distance from the apparent critical-angle transition to the nearest guided-mode location. The choice of this distance depends at least in part on the shape of the spike region R1, and may be chosen based on empirical evidence from data collected on multiple IOX articles 10.

In another example, the determination of whether both the TM and TE mode spectra 113TM and 113TE are within a preferred measurement window is based on the relationship between the second derivative of the intensity profile of the nearest mode (fringe) to the critical index and the distance between this nearest mode and the apparent location of the critical angle transition 116. Qualitatively, the same method applies for analyzing this relationship for a bound mode and for a leaky mode, except that the decision threshold for a bound mode need not be the same as for a leaky mode.

In some embodiments, the distance between the offending mode and the apparent critical-angle transition 116 is compared to a numerical factor divided by the square root of the second derivative of the optical intensity at the mode location. This is based on the observation that the full width at half-maximum (FWHM) for many bell-shaped intensity distributions of resonant peaks of unit peak value is proportional to the inverse of the square root of the second derivative of the intensity at the location of the resonance (at the minimum for an intensity dip or at the maximum for an intensity peak).

For example, for a Lorentzian of unit peak value, the FWHM is about

$$\frac{5.66}{\sqrt{{I}^{*}}} ,$$

for a Gaussian of unit-peak value it is about

$$\frac{2.35}{\sqrt{{I}_{2}^{*}}} ,$$

and for a hyperbolic secant it is about

$$\frac{2.63}{\sqrt{{I}^{*}}} ,$$

where I* stands for the second derivative of intensity with respect to the horizontal variable of the spectrum (for example, position, angle, effective index, or point number). In many cases, the apparent position of the critical-angle transition 116 is substantially unaffected by the nearby (nearest) mode if the distance between the transition and the nearby mode is larger than about 1.8 times the FWHM breadth of the resonance of the nearby mode.

In some embodiments, the measured mode spectrum 113TM or 113TE is considered outside the preferred measurement window when the distance between the location of a nearby mode and the (apparent) critical-angle transition 116 for the same polarization state is less than 1.8 times the FWHM breadth of the coupling resonance of the nearby mode.

In some embodiments, measured mode spectrum 113TM or 113TE is considered outside the preferred measurement window if it is within less than 1.5 times the FWHM breadth of the coupling resonance of said nearby mode, such as less than 1.2, less than 1, less than 0.8, less than 0.6, or less than 0.5 times the FWHM breadth of the coupling resonance of said nearby mode.

A preferred threshold ratio for the determining whether a measured mode spectrum 113TM or 113TE is inside or outside a preferred measurement window can be based on a trade-off between the importance of high precision for the measurement of the given stress parameter (e.g., knee stress CSₖ) and the importance of having a broad measurement window. Greater importance on measurement accuracy favors a larger ratio of the minimum acceptable spacing to the FWHM, and vice versa.

In addition, in cases where the shape of the intensity distribution corresponding to the given mode is well-described by a Lorentzian profile, a preferred threshold value of the ratio maybe higher, such as in the range of 0.8 to 1.8. In cases where the given mode is well-described by a Gaussian profile, the preferred threshold value of the ratio may be lower, such as in the range of 0.5 to 1.2.

Based on the above considerations, a measured mode spectrum 133TM or 113TE is deemed outside the preferred measurement window when the distance between the apparent position of the critical-angle transition 116 and the nearby offending mode is less than or equal to about

$$\frac{10.2}{\sqrt{{I}^{*}}} .$$

This is a relatively strict criterion used to ensure at most a negligible shift in the apparent position of the critical-angle transition 116. In various cases, of different line shapes and preferred trade-off between the target accuracy of the stress parameter in question (e.g., knee stress CSₖ) and breadth of the preferred measurement window, a less-strict threshold for the spacing maybe chosen. For example, the spacing may be less than or equal to a factor of 8.5, such as less than or equal to a factor of 6.8, 5.7, 4.5, 3.4, or 2.8 times

$$\frac{1}{\sqrt{{I}^{*}}} .$$

Furthermore, in some cases where the shape of the nearby mode resonance is far from Lorentzian, and closer to Gaussian, and a maximized breadth of the measurement window is a higher priority, the preferred threshold value of the spacing between the mode and the apparent transition position 116 may be less than or equal to 2.4, such as less than or equal to 1.9, 1.4, or 1.2 times

$$\frac{1}{\sqrt{{I}^{*}}} .$$

The second derivative at the location of the nearby mode may be found by smoothing of the signal by low-pass filtering, finding a first derivative digitally and smoothing it by low-pass filtering, then finding a second derivative digitally, smoothing it, and taking the value at the location of the mode resonance. In some embodiments, the second derivative may be found by fitting a parabola (second-order polynomial) to the signal in the closest vicinity of the mode location, and taking the second derivative of the fitting parabola to serve as the second derivative representing the coupling resonance of the mode. Such methods for finding the second derivative are known in the art.

Furthermore, in some embodiments, the intensity distribution in the vicinity of a mode resonance (guided or leaky mode) is normalized so that the minimum intensity corresponds to 0 and the maximum intensity corresponds to 1, or vice versa. In one example of a reflection mode spectrum where the maximum coupling on resonance with a guided or leaky mode corresponds to a local minimum in the reflected intensity, the minimum intensity at the bottom of the reflected-intensity dip may be subtracted from the entire intensity distribution so that a second intensity distribution has a minimum at 0. Then the second intensity distribution is multiplied by a scaling factor so that the maximum value in the vicinity of the local minimum becomes equal to 1. This provides a scaled normalized intensity distribution having a range from 0 to 1. The second-derivative may then be calculated after the normalization procedure.

If it is found that both of the measured TM and TE mode spectra 113TM and 113TE are in the preferred measurement window, then the knee stress CSₖ and related parameters (e.g., depth of spike D1, depth of layer DOL, etc.) can be determined using these mode spectra. Furthermore, if the TM mode spectrum 113TM is found to be within the preferred measurement window, it may be chosen to calculate the depth of layer DOL based on the TM fringe count only before deciding whether to determine the knee stress CSₖ using the same TM mode spectrum and the associated TE spectrum 113TE that was measured at the same time.

If it is found that one of the measured TM and TE mode spectra 113TM or 113TE resides outside of the preferred measurement window, then another (second) pair of TM and TE mode spectra 113TM or 113TE is considered. The second pair of mode spectra 113TM and 113TE may be collected after the determination was made that the at least one of the first TM and TE spectrum was not inside the preferred measurement window. Alternatively, the second pair of mode spectra 113TM and 113TE can be collected in advance using the prism-coupling system 28 set to different measurement conditions than used in obtaining the first pair of mode spectra.

In an example, the light source system 60 of the prism-coupling system 28 is adjusted so that the light 62 has a different wavelength for the second measurement than the first measurement. The second wavelength may be chosen to provide continuity of the preferred measurement window so that as an IOX article 10 that falls barely outside the preferred measurement window for the first wavelength falls inside the preferred measurement window for the second spectrum having a different wavelength.

For example, consider the IOX article formed from a Li-containing aluminosilicate glass-based substrate 20 and with a spike region R1 formed using a K⁺ IOX process. The measured mode spectrum 113TM or 113TE has full mode count between about 2.1 and about 3 fringes at a first measurement wavelength of 590 nm. The calculated surface compressive stress is the range of 500 to 900 MPa.

This particular example IOX article 10 can benefit from a second measurement of the mode spectra 113TM and 113TE using a second wavelength that is longer than the first wavelength by between about 1% and 15% to shift the fringe count range inside a preferred process (measurement) window having a range on the full mode count of 2.3 to 2.7.

Similarly, when a measured mode spectra 113TM or 113TE yield a mode count is just below the lower end of the preferred measurement window (for the present case, when the fringe count falls in the range 1.75-2.1 fringes), then the second wavelength may be made shorter by between about 1% and 25%, depending on how far the mode fringe count falls outside the preferred measurement window.

A more significant shift of the preferred measurement window can be used by making a larger wavelength shift, such as by 18%, 25%, or 30%. A larger shift in the measurement wavelength can be used to establish a larger measurement window by combining the measurement windows of two different measurement wavelengths.

In an example a condition where a spike requires a wavelength that is between two neighboring measurement wavelengths to fall inside the preferred measurement window is avoided. In an example, for a spike having a linear shape with surface index increment Δn above the base index n, the relationship between the fringe count N, the measurement wavelength λ, and the spike depth D1 or DOLₛₚ is:

$$N \approx 3.77 \sqrt{n Δ n} \frac{{DOL}_{sp}}{λ} + 0.25$$

The difference in fringe count between the TM and the TE mode spectrum 113TM and 113TE depends on the difference in between the two mode spectra, since the other parameters that determine the fringe count are the same for the two polarization states in the measurement. If the surface compressive stress is labeled CS, and the knee stress is CSₖ, then the difference in between the two polarizations is approximately equal to (CS-CSₖ)/SOC, where SOC is the stress-optic coefficient. The SOC is typically within 15% of 3×10⁻⁶ RIU/MPa for most chemically strengthened glasses, where RIU stands for refractive-index units.

For a spike produced by an IOX process using K in a Na-based or Li-based glass substrate 20, the difference in Δn between TM and TE is usually about 1/5.6 of the average of the two Δn values. If the stress-induced birefringence of Δn is labeled δn^{TM-TE}, then the difference in fringe count between the two polarizations is:

$$δ {N}^{TM - TE} \approx 3.77 \frac{{DOL}_{sp}}{λ} \sqrt{n} ( \sqrt{Δ {n}^{TM}} - \sqrt{Δ {n}^{TE}} )$$

$$\frac{δ {N}^{TM - TE}}{{N}^{TM} - 0.25} \approx \frac{( \sqrt{Δ {n}^{TM}} - \sqrt{Δ {n}^{TE}} )}{\sqrt{Δ {n}^{TM}}} = \frac{1}{\sqrt{Δ {n}^{TM}}} \frac{Δ {n}^{TM} - Δ {n}^{TE}}{\sqrt{Δ {n}^{TM}} + \sqrt{Δ {n}^{TE}}} \approx \frac{Δ {n}^{TM} - Δ {n}^{TE}}{2 Δ {n}^{TM}} \approx \frac{1}{11.2} \approx 0.09$$

This means that the fringe count for the TE polarization state is typically about 10/11 of the fringe count of the TM polarization state. Therefore, the fringe count for TE is different by:

$$δ {N}^{TM - TE} \approx \frac{{N}^{TM} - 0.25}{11.2} \approx 0.09 {N}^{TM} - 0.022$$

The factor of 0.09 relating the mode count difference to the TM mode count will vary slightly with variations in SOC, and is approximately proportional to the square root of the ratio of SOC to 3×10⁻⁶. For SOC ranging from about 2×10⁻⁶ to about 4.5×10⁻⁻⁶, the corresponding factor would vary from about 0.073 to about 0.11.

Having established that a preferred measurement window for each polarization has a fractional part FP of the fringe count between about 0.1 and 0.8, such as between about 0.15 and 0.75, a preferred measurement window for each polarization may span about 0.6 fringes. Given that there is an offset between the TM and the TE fringe count, the effective preferred measurement window for simultaneously having an accurate measurement of the critical index in the TM and the TE polarization is reduced compared to the single-polarization preferred measurement window by the difference in mode count between TM and TE.

In an example for a typical glass with 0.073N^{TM}≤δN^{TM-TE}≈0.11N^{TM}, for a TM mode spectrum 113TM having about 2.6 TM fringes 115TM, the preferred measurement window is reduced from about 0.6 fringes for TM polarization alone to 0.6−(0.19 to 0.29)=(0.31 to 0.41) fringes. Similarly, for a target TM mode spectrum 113TM having 3.6 TM fringes 115TM, the preferred measurement window is reduced from about 0.6 to about 0.6−(0.26 to 0.40)=(0.2 to 0.34) fringes. In the former case, the reduction is between about ⅓ and ½, depending on the value of SOC, while in the latter case the reduction is approximately from about ½ to about ⅔ of the single-polarization preferred window. Thus, the offset in fringe count between TM and TE mode spectra 113TM and 113TE substantially reduces the effective breadth of the continuous process window available within a single preferred measurement window.

In some embodiments where the first TM or TE spectrum 113TM or 113TE measured at a first measurement wavelength fails to fall inside the preferred measurement window, then a mode spectrum measured at a different (second) measurement wavelength is used to position the TM and the TE spectrum inside the preferred measurement window. If the mode spectrum having a larger fringe count (usually the TM spectrum 113TM) has between about 2.75 and 3.15 fringes, then the measurement wavelength may be increased to bring the fringe count for the TM spectrum in the preferred range 2.15-2.75.

To shift the entire uncovered range 2.75-3.15 fringes to the preferred measurement window 2.15-2.75 fringes using a single longer wavelength, it may be preferred that the single longer second wavelength be at least 12% longer than the first measurement wavelength, preferably 14% longer or more.

On the other hand, it may be desirable to ensure continuity of the measurement so that no IOX article that at the first measurement wavelength has between 2.75 and 3.15 fringes in the polarization state with higher fringe count also falls outside the preferred measurement window at the second (longer) wavelength. Thus, for the longer second measurement wavelength, it may be preferred that the fringe count in the other polarization state does not fall out of the preferred measurement window. In the present example, the change in wavelength is used to shift the mode count from the range 2.15-2.75 fringes to below about 2.1 fringes.

In an example, the higher-fringe-count polarization may have 2.6-2.75 fringes at the first wavelength, and the lower-fringe-count polarization may have 2.35-2.55 fringes for a typical glass with SOC of about

$$3 \times {10}^{- 6} \frac{RIU}{MPs} .$$

Then, for the example with a lower fringe count of 2.35, a wavelength increase beyond 12% would cause said lower fringe count to drop below 2.1, falling out of the preferred measurement window.

On the other hand, for a mode fringe count of 2.55, a wavelength increase of up to 19.6% would retain the corresponding mode spectrum within the extended preferred measurement window of 2.1-2.8 fringes. Hence, for a typical glass substrate, it is preferred that the wavelength change for the second wavelength not exceed 20% of the first wavelength, such as not exceed 12% of the first wavelength.

In some embodiments, it is preferable that a continuous capability of measuring an IOX article 10 inside a preferred measurement window available among the two or more wavelengths instead of gaining the maximum possible extension of the preferred measurement window by covering the entire problematic range of 2.75-3.15 fringes by switching to the longer wavelength. Hence, having a wavelength increase exceeding 12% or 14% of the first wavelength can be desirable but may not be required or strongly preferred. On the other hand, having a wavelength increase below 20% for some glasses or below 12% for most glasses may be strongly preferred to enable continuous availability of a preferred measurement window among a large variety of IOX articles 10 centered around a target measurement spectrum with 2.1-2.8 fringes. There are less common glasses with a SOC that is substantially lower, such as in the range

$$0.5 \times {10}^{- 6} to 2 \times {10}^{- 6} \frac{RIU}{MPs} ,$$

for which significantly larger wavelength increase is possible without falling out of the preferred measurement window for the polarization having the lower fringe count.

Examples of preferred wavelength changes for 4 different values of the stress-optic coefficient measured in Brewsters, or B

$$( 1 B = {10}^{- 6} \frac{RIU}{MPs} )$$

are given in Tables 1 through 4, below. These examples are given for the case where the preferred change is to increase the wavelength because the larger of the two fringe counts is exceeding the upper end of the measurement window. When the smaller fringe count falls below the bottom of the preferred measurement window, the preferred change is to a shorter wavelength, and similar wavelength percentage changes would be preferable as in the examples of Tables 1 through 4.

Table 1 provides the preferred wavelength change for measurement windows with different fringe counts, for a material with a SOC of about 1 B.

TABLE 1

| | | | | | | | | | desirable | |
|---|---|---|---|---|---|---|---|---|---|---|
| | | | | | | | preferred | | wavelength | low |
| | | | | | min | | min | preferred | increase for | count |
| Preferred | larger | | fringe | smaller | smaller | max | smaller | max | maximum | limits |
| window | fringe | SOC | count | fringe | fringe | wavelength | fringe | wavelength | measurement | max |
| fringes | count | (B) | diff. | count | count | increase % | count | increase % | window | shift? |
| 2-3 | 2.75 | 1 | 0.07 | 2.68 | 2.1 | 31.1 | 2.15 | 27.7 | 16.0 | N |
| 3-4 | 3.75 | 1 | 0.10 | 3.65 | 3.1 | 19.2 | 3.15 | 17.1 | 11.4 | N |
| 4-5 | 4.75 | 1 | 0.13 | 4.62 | 4.1 | 13.4 | 4.15 | 12.0 | 8.9 | N |
| 5-6 | 5.75 | 1 | 0.16 | 5.59 | 5.1 | 10.0 | 5.15 | 8.9 | 7.3 | N |
| 6-7 | 6.75 | 1 | 0.19 | 6.56 | 6.1 | 7.8 | 6.15 | 6.9 | 6.2 | N |
| 7-8 | 7.75 | 1 | 0.22 | 7.53 | 7.1 | 6.2 | 7.15 | 5.5 | 5.3 | N |
| 8-9 | 8.75 | 1 | 0.25 | 8.50 | 8.1 | 5.1 | 8.15 | 4.4 | 4.7 | Y |
| 9-10 | 9.75 | 1 | 0.28 | 9.47 | 9.1 | 4.1 | 9.15 | 3.6 | 4.2 | Y |
| 10-11 | 10.75 | 1 | 0.31 | 10.44 | 10.1 | 3.4 | 10.15 | 2.9 | 3.8 | Y |
| 11-12 | 11.75 | 1 | 0.34 | 11.41 | 11.1 | 2.8 | 11.15 | 2.4 | 3.5 | Y |
| 12-13 | 12.75 | 1 | 0.37 | 12.38 | 12.1 | 2.3 | 12.15 | 1.9 | 3.2 | Y |
| 13-14 | 13.75 | 1 | 0.40 | 13.35 | 13.1 | 1.9 | 13.15 | 1.5 | 3.0 | Y |

Table 2 provides the preferred wavelength change for measurement windows with different fringe counts, for a material with SOC of about 2 B.

TABLE 2

| | | | | | | | | | desirable | |
|---|---|---|---|---|---|---|---|---|---|---|
| | | | | | | | preferred | | wavelength | low |
| | | | | | min | | min | preferred | increase for | count |
| Preferred | larger | | fringe | smaller | smaller | max | smaller | max | maximum | limits |
| window | fringe | SOC | count | fringe | fringe | wavelength | fringe | wavelength | measurement | max |
| fringes | count | (B) | diff. | count | count | increase % | count | increase % | window | shift? |
| 2-3 | 2.75 | 2 | 0.15 | 2.60 | 2.1 | 27.1 | 2.15 | 23.7 | 16.0 | N |
| 3-4 | 3.75 | 2 | 0.21 | 3.54 | 3.1 | 15.5 | 3.15 | 13.5 | 11.4 | N |
| 4-5 | 4.75 | 2 | 0.27 | 4.48 | 4.1 | 9.9 | 4.15 | 8.5 | 8.9 | Y |
| 5-6 | 5.75 | 2 | 0.33 | 5.42 | 5.1 | 6.7 | 5.15 | 5.6 | 7.3 | Y |
| 6-7 | 6.75 | 2 | 0.39 | 6.36 | 6.1 | 4.5 | 6.15 | 3.6 | 6.2 | Y |
| 7-8 | 7.75 | 2 | 0.45 | 7.30 | 7.1 | 3.0 | 7.15 | 2.2 | 5.3 | Y |
| 8-9 | 8.75 | 2 | 0.51 | 8.24 | 8.1 | 1.8 | 8.15 | 1.2 | 4.7 | Y |
| 9-10 | 9.75 | 2 | 0.57 | 9.18 | 9.1 | 1.0 | 9.15 | 0.4 | 4.2 | Y |

Table 3 provides the preferred wavelength change for measurement windows with different fringe counts, for a material with SOC of about 3 B.

TABLE 3

| | | | | | | | | | desirable | |
|---|---|---|---|---|---|---|---|---|---|---|
| | | | | | | | preferred | | wavelength | low |
| | | | | | min | | min | preferred | increase for | count |
| Preferred | larger | | fringe | smaller | smaller | max | smaller | max | maximum | limits |
| window | fringe | SOC | count | fringe | fringe | wavelength | fringe | wavelength | measurement | max |
| fringes | count | (B) | diff. | count | count | increase % | count | increase % | window | shift? |
| 2-3 | 2.75 | 3 | 0.22 | 2.53 | 2.1 | 23.1 | 2.15 | 19.8 | 16.0 | N |
| 3-4 | 3.75 | 3 | 0.31 | 3.44 | 3.1 | 11.8 | 3.15 | 9.9 | 11.4 | Y |
| 4-5 | 4.75 | 3 | 0.40 | 4.35 | 4.1 | 6.4 | 4.15 | 5.1 | 8.9 | Y |
| 5-6 | 5.75 | 3 | 0.49 | 5.26 | 5.1 | 3.3 | 5.15 | 2.2 | 7.3 | Y |
| 6-7 | 6.75 | 3 | 0.58 | 6.17 | 6.1 | 1.2 | 6.15 | 0.3 | 6.2 | Y |

Table 4 provides the preferred wavelength change for measurement windows with different fringe counts, for a material with SOC of about 4 B.

TABLE 4

| | | | | | | | | | desirable | |
|---|---|---|---|---|---|---|---|---|---|---|
| | | | | | | | preferred | | wavelength | low |
| | | | | | min | | min | preferred | increase for | count |
| Preferred | larger | | fringe | smaller | smaller | max | smaller | max | maximum | limits |
| window | fringe | SOC | count | fringe | fringe | wavelength | fringe | wavelength | measurement | max |
| fringes | count | (B) | diff. | count | count | increase % | count | increase % | window | shift? |
| 2-3 | 2.75 | 4 | 0.30 | 2.45 | 2.1 | 19.0 | 2.15 | 15.9 | 16.0 | N |
| 3-4 | 3.75 | 4 | 0.42 | 3.33 | 3.1 | 8.2 | 3.15 | 6.3 | 11.4 | Y |
| 4-5 | 4.75 | 4 | 0.54 | 4.21 | 4.1 | 3.0 | 4.15 | 1.6 | 8.9 | Y |

The examples in Tables 1 through 4 demonstrate that in some cases it can be advantageous to change the wavelength up to about 28% of the first measurement wavelength. In many cases, the main benefits can be obtained with significantly smaller wavelength changes, e.g., in the range of 8-24%. Mode spectra containing more fringes per polarization state require smaller wavelength shifts to achieve simultaneous preferred-window condition for both wavelengths. For such cases, several discrete wavelengths (3 or more) may be required to provide a wide enough fabrication window with continuous accurate quality control measurement coverage.

Example IOX Article

In one example, an IOX article 10 was formed from a glass substrate 20 having the composition 63.16 mol % SiO₂, 2.37 mol % B₂O₃, 15.05 mol % Al₂O₃, 9.24 mol % Na₂O, 5.88 mol % Li₂O, 1.18 mol % ZnO, 0.05 mol % SnO₂, and 2.47 mol % P₂O₅, and a SOC of about 3B. A DIOX process for chemical strengthening was employed. After a first K⁺-L⁺ IOX step (i.e. with r as the in-diffusing ion 11), the TM and TE mode spectra 115TM and 115TE each had between 2 and 3 fringes at a first measurement wavelength λ=590 nm. After a second IOX step, the TM and TE mode spectra 115TM and 115TE each had between 3 and 4 fringes at 590 nm. The surface stress CS associated with the formation of the K⁺-based spike region R1 is usually in the range 500 to 640 MPa. The surface stress CS after the second IOX step using Na+ as the in-diffusing ion 12 is typically in the range 750-950 MPa.

Using the methods described herein, the measurement requirements for both step 1 and step 2 can be fully met with a continuous effective preferred measurement window when using three measurement wavelength windows centered around the measurement wavelengths λ of 545 nm, 590 nm, and 640 nm, respectively. Furthermore, in an example it is preferred that the spectral bandwidth of the measurement light 62 not exceed about 8 nm, 9 nm, and 10 nm, respectively, at these measurement wavelengths. For even higher fringe contrast, the spectral bandwidths can be limited to 4 nm, 5 nm, and 6 nm, respectively. Thus, in one example, each measurement wavelength has a spectral band of 10 nm or less, or in another example, of 6 nm or less.

When the fringe count is close to either edge of the 590 nm measurement window after step 2, the mode spectrum is brought back inside the preferred measurement window by either increasing or decreasing the measurement wavelength, depending on whether the upper or the lower end of the measurement window approaches at 590 nm. In another example using a three measurement wavelength implementation, the shortest measurement wavelength is about 540 nm, the middle measurement wavelength is about 595 nm, and the longest measurement wavelength is about 650 nm.

While two or three measurement wavelengths have been discussed above by way of example, any reasonable number of measurement wavelengths may be used. For example, using two measurement wavelengths can increase the measurement window by up to a factor of 2, and may be quite adequate to satisfy the needs for a reasonable fabrication process window. On the other hand, in some cases where the spike depth D1 is relatively is large and produces several (e.g., 3, 4, or more) fringes per polarization state, or when the SOC is very high such as 4 B, more than three wavelengths may be preferred. The multiple measurement wavelengths can be positioned closer together than in the three wavelength examples above, e.g., spaced by 7.6% and 9.2%, respectively, of the average wavelength which in these examples is the middle of the three measurement wavelengths.

An exemplary method suppresses systematic errors in the measurement of the knee stress CSₖ and in the measurement of the depth of the spike DOLₛₚ. The suppression of systematic errors may be essentially complete when the multiple measurement wavelengths are carefully chosen to be close enough to allow a seamless transition between preferred measurement windows at the different wavelengths. This means that the preferred measurement windows at neighboring wavelengths can overlap at least slightly.

The examples listed in Tables 1 through 4 allow selecting preferable wavelength shifts that guarantee such overlap and measurements that are substantially free of systematic errors for a range of samples that may cover a continuous range of fabrication conditions. On the other hand, when the wavelengths are spaced slightly more than the preferred spacing that guarantees window overlap, a maximum expansion of measurement capability is obtained, but at the expense of only partial suppressing systematic errors. The possibility still exists that certain IOX articles 10 can show deviations from accurate measurement, even though the probability of having such samples decreases due to the much increased coverage of the production range with multiple preferred measurement windows.

In some embodiments, the corrective action taken when at least one of the TM and TE spectra 113TM and 113TE is/are not in the preferred measurement window includes changing the thickness of the interfacing fluid 52 (e.g., index oil) to help bring the problematic spectrum inside a preferred measurement window. This is possible because the interfacing fluid can be considered part of the waveguide 26. The main problem that is solved with this corrective action is to determine the knee stress CSₖ correctly, i.e., to within select tolerance. The preferred refractive index of the interfacing fluid 52 at the measurement wavelength is higher than the critical index for the polarization state in which the problematic spectrum occurs. Furthermore, the preferred refractive index of the interfacing fluid 52 is higher than the critical index by no more than 0.1, such as by no more than about 0.06, or by no more than 0.04. In some embodiments, the interfacing fluid 52 can be selected to have a refractive index as close as possible to the expected refractive index on the surface of the glass (e.g., the surface refractive index of the potassium spike).

In particular, the interfacing fluid refractive index n_{f} may be within about 0.004 or 0.003 of the surface refractive index no. The surface refractive index n₀ is usually different for TM and TE polarizations due to the significant surface stress in the spike, but the difference is usually less than 0.004, and most often less than 0.003. As noted above, the interfacing fluid 52 resides between the prism coupling surface 44 of the coupling prism 40 and the surface 12 of the IOX article 10, and the thickness TH of the interfacing fluid can be controlled using the vacuum system 56. Initially, the amount vacuum can be relatively high, making the thickness TH of the interfacing fluid 52 relatively small, e.g., 200 nm or less, or even 100 nm or less. With this thickness TH of the interfacing fluid 52, the surface compressive stress CS and the spike depth D1 can be measured with adequate accuracy. The spike depth D1 may be over-estimated by as much as 0.1 microns, or even 0.2 microns, which may be acceptable in many cases. The surface compressive stress CS may be slightly under-estimated by assuming that the interfacing fluid thickness is 0 when in fact it might be as high as 0.1 or even 0.2 microns.

In an example, the thickness TH of the interfacing fluid 52 is adjusted in a way that it increases the effective index of a leaky mode to turn it to a quasi-guided mode of the waveguide 26, wherein a quasi-guided mode has an effective index higher than that of the index corresponding to the critical-angle transition.

In another example, thickness TH of the interfacing fluid 52 is adjusted in a way that increases the effective index of a leaky mode to turn it to a quasi-guided mode the waveguide 26 so that fractional part FP of the new mode count now falls in the preferred (extended) measurement window MWE, wherein the refractive index of the interfacing fluid may be higher than the refractive index corresponding to the critical angle.

In another example, the thickness TH of the interfacing fluid 52 is adjusted to decrease the effective index of a leaky mode to turn it into a quasi-guided mode of the waveguide 26 so that fractional part FP of the new mode (fringe) count now falls in the preferred measurement window MWE. In this case, the index of the interfacing fluid 52 may be lower than the index corresponding to said critical angle.

Another example includes changing the refractive index of the interfacing fluid 52 to change the effective index of the leaky mode to turn it to a quasi-guided mode having an effective index higher than the critical-angle effective index. The example can also include changing the fractional part FP of the fringe count so that the fractional part FP falls within a fractional-part range associated with the preferred measurement window. In the description herein, changing the refractive index of the interfacing fluid 52 includes replacing at least a portion of a first interfacing fluid having a first refractive index with a second interfacing fluid having a second refractive index. This process can be used to define essentially any refractive index between the first refractive index and the second refractive index.

Once the prism-coupling system 28 is placed in the desired configuration and the mode spectrum 113 collected, the CS and DOL values are then recorded. If both the TM and the TE mode spectra 113TM and 113TE fall within in the preferred measurement window as described above, then the TM and TE critical index n_{crit} is measured by the location of the highest slope in the intensity profiles of the respective critical angle transitions 116. This provides a measure of the birefringence, which is used to calculate the knee stress CSₖ.

On the other hand, if at least one of the TM or TE mode spectra 115TM and 115TE is not in the preferred measurement window, then a leaky or a guided mode in the problematic TM or TE mode spectrum may be offending, i.e., has an effective index too close to the critical index and adversely affects the apparent location of the critical-angle transition 116. At this point, the thickness TH of the interfacing fluid 52 can be increased, e.g., by decreasing the vacuum (e.g., increasing the pressure) until the effective index of the problematic leaky or guided mode increases enough to be non-offending, i.e., becomes far enough above the critical index that the critical-angle transition 116 is substantially undisturbed and the critical angle (and hence the critical index) for the given polarization can be accurately measured.

It would be preferred that the critical angles for both the TM and TE polarizations be measured at the same time, but this is not required. If the first measured mode spectrum for the other polarization state was in the preferred measurement window before taking the corrective action, it is possible to measure the CSₖ by using the measured critical-angle position for the other polarization state using the original thickness of the index-matching fluid 52. Choosing to take both measurements of the TM and TE mode spectra 113TM and 113 TE at the same time helps avoid errors from slight changes in the prism-coupling system 28 that can occur over time.

Experimental Results

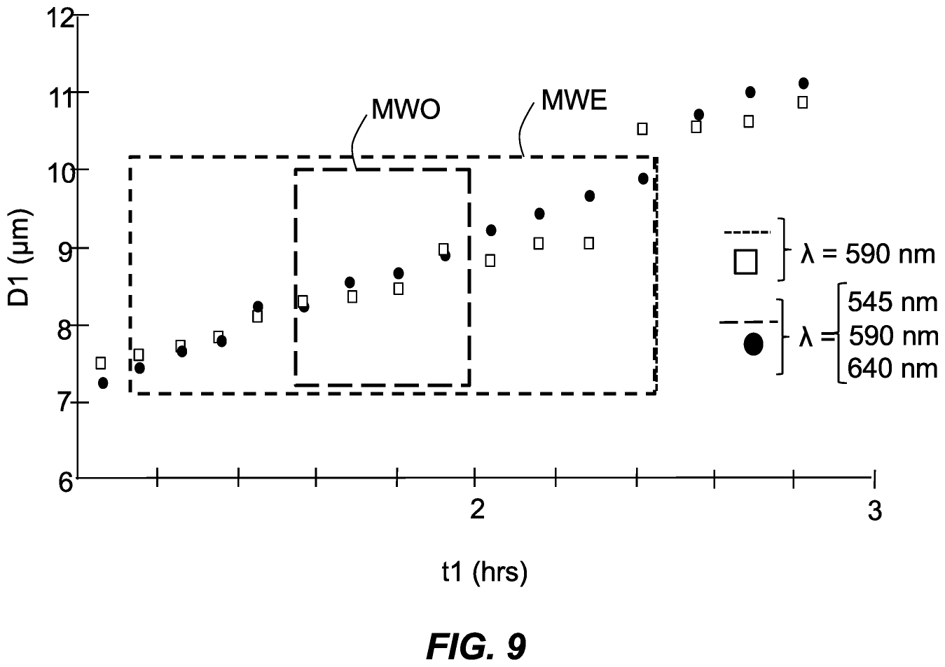

FIG. 9 is a plot of the spike depth D1 (μm) versus time t1 (hours) for a first IOX process for an IOX article 10 made using a lithium-based aluminosilicate glass substrate 20. The open squares are measurements made using the prism-coupling system 28 with a light source system 60 operating at a single measurement wavelength λ of 595 nm. The dark circles are measurements with the prism-coupling system 28 with a light source system 60 configured to operate at three different measurement wavelengths λ centered at 540 nm, 595 nm, and 650 nm.

An initial (“original”) measurement window MWO for the single-wavelength measurement method is depicted with long-dash lines, while an extended (preferred) measurement window MWE for the three-wavelength measurement methods and prism-coupling systems as described herein is shown with short-dash lines. The extended measurement window MWE that uses three measurement wavelengths λ is significantly extended as compared to the single-wavelength measurement window MWO. Since the IOX process time defines the refractive index profile of the IOX article 10, an extended measurement window MWE having a wider range of IOX process times means IOX articles with a larger range of spike-based refractive index profiles can be characterized for at least one stress characteristic such as the knee stress CSₖ.

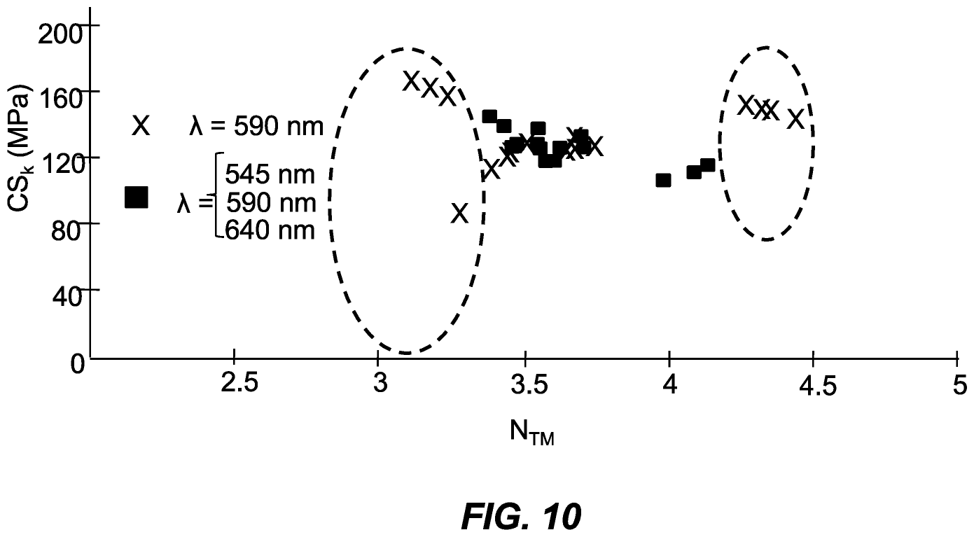

FIG. 10 is a plot of the knee stress CSₖ (MPa) versus the TM mode (fringe) count N_{TM} for example IOX articles 10 formed from lithium-containing aluminosilicate glass substrates 20. The single-wavelength measurements were made at a measurement wavelength λ of 595 nm and are represented by x's. Three-wavelength measurements were made at measurement wavelengths of 540 nm, 595 nm, 650 nm and are represented by dark squares. The single-wavelength measurements do not follow a monotonic continuous decrease of CSₖ with increased mode count, while the three-wavelength measurements follow such a pattern within a precision limited by a relatively small measurement noise not exceeding 20 MPa. The increase in mode count was obtained with a two-step IOX process, where step 1 was the same for all samples, and in step 2 the diffusion time was varied between different samples, all using the same step 2 IOX bath. The TM mode (fringe) count varies between about 3 and about 4.5 modes in this data set. The TE mode count is lower than the TM mode count by about 9% (0.3-0.4 fringes) for the same data set and is outside the extended measurement window for the encircled data points.

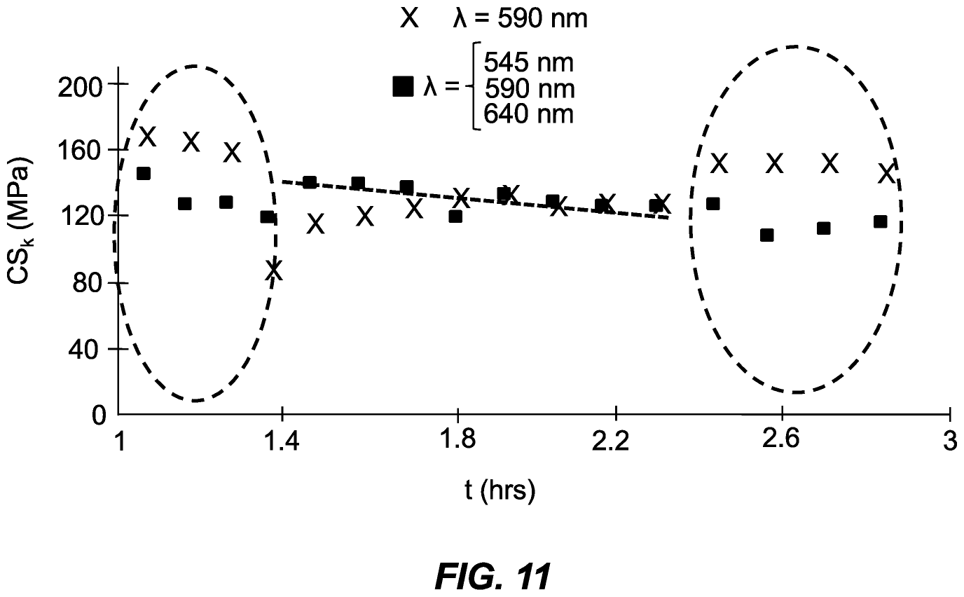

FIG. 11 plots the measured knee stress CSₖ after the two-step ion exchange (DIOX) versus the diffusion (ion-exchange) time t (hours) for the same IOX articles 10 measured in FIG. 5. The expected trend of CSₖ is a slow monotonic decrease with increasing time t. The single-wavelength (λ=595 nm) measurements are shown with x-symbols, while the three-wavelength (λ=540 nm, 595 nm and 650 nm) measurement results are shown as dark squares. Even though the three wavelengths are somewhat farther apart than optimum for continuous coverage, the data points for the three-wavelength measurements better hew to the expected monotonic trend (dashed line) than the single-wavelength measurements. Thus, the prism-coupling system 28 having a light source system 60 that emits two or more closely spaced measurement wavelengths (e.g., λ=545 nm, 590 nm, and 640 nm) significantly reduces the deviations from the expected monotonic trend, which translates into more accurate measurement of stress-related characteristics.

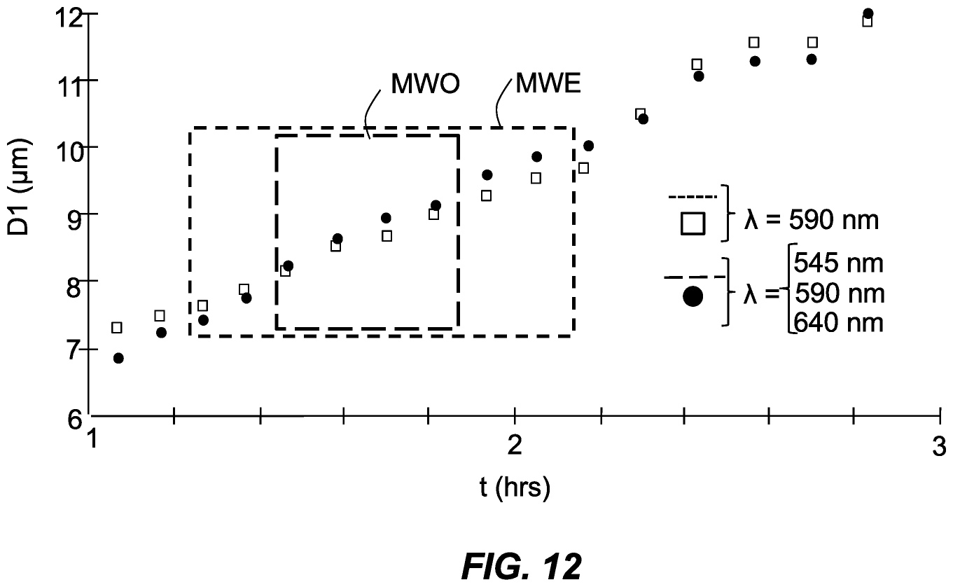

FIG. 12 is similar to FIG. 4 and plots the spike depth D1 (microns) versus time t (hours) for the IOX articles 10 of FIG. 6 for measurements made using a single-wavelength prism-coupling system 28 having a single measurement wavelength of 595 nm (open squares), and a prism-coupling system 28 having three measurement wavelengths of 540 nm, 595 nm, and 650 nm (dark circles). The single-wavelength measurement window MWO is shown with long-dash lines, while the extended measurement window MWE is shown with short-dash lines. On the upper right edge of the preferred measurement window the reported spike depth D1 falls below the accurate value due to the proximity of an offending TM leaky mode to the TM critical angle transition 116. Likewise, on the lower left edge of the preferred measurement window, an offending TE guided mode occurs too close to the critical angle transition 116. The knee stress CSₖ can be under-estimated even though the spike depth D1 is not affected since the spike depth D1 in the plots is measured based on the TM mode spectrum 113 TM only. A more accurate estimation of the knee stress can be obtained by including data from both the TE and TM mode spectra 113TE and 113TM.

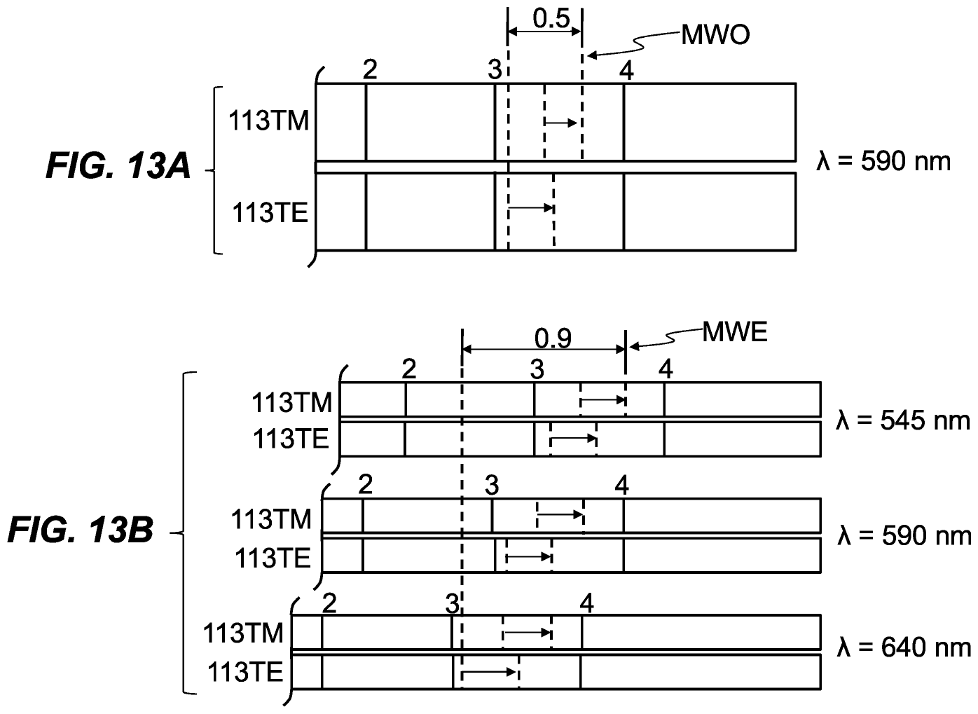

FIGS. 13A and 13B are schematic diagrams of the TM and TE mode spectra 113TM and 113TE that respectively include four TM and TE modes or fringes 115TM and 115TE. FIG. 13A shows mode spectra 113TM and 113TE for a single measurement wavelength while FIG. 13B shows three pairs of mode spectra 113TM and 113TE, one pair for each of three measurement wavelengths of 545 nm, 590 nm and 640 nm. The effective measurement window MWO for the single-wavelength system of FIG. 13A has a size of about 0.5 fringes while the extended measurement window MWE for the three-wavelength system of FIG. 13B measured is about 0.9 fringes, or about twice that of the single-wavelength measurement window MWO.

Frangibility

Frangible behavior or “frangibility” refers to specific fracture behavior when a glass-based article is subjected to an impact or insult. As utilized herein, a glass-based article (and in particular, a glass-based IOX article 10 such as considered herein) is considered non-frangible when it exhibits at least one of the following in a test area as the result of a frangibility test: (1) four or less fragments with a largest dimension of at least 1 mm, and/or (2) the number of bifurcations is less than or equal to the number of crack branches. The fragments, bifurcations, and crack branches are counted based on any 2 inch by 2 inch square centered on the impact point. Thus, a glass-based article is considered non-frangible if it meets one or both of tests (1) and (2) for any 2 inch by 2 inch square centered on the impact point where the breakage is created according to the procedure described below. In various examples, the chemically strengthened IOX article 10 can be frangible or non-frangible.

In a frangibility test, an impact probe is brought into contact with the glass-based article, with the depth to which the impact probe extends into the glass-based article increasing in successive contact iterations. The step-wise increase in depth of the impact probe allows the flaw produced by the impact probe to reach the tension region while preventing the application of excessive external force that would prevent the accurate determination of the frangible behavior of the glass. In one embodiment, the depth of the impact probe in the glass may increase by about 5 μm in each iteration, with the impact probe being removed from contact with the glass between each iteration. The test area is any 2 inch by 2 inch square centered at the impact point.

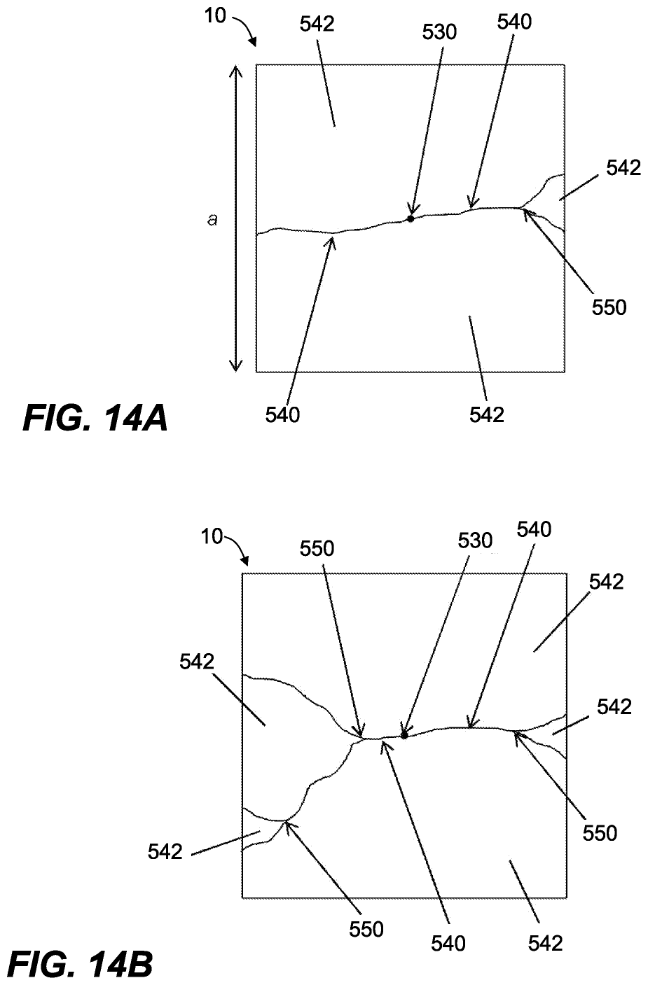

FIG. 14A depicts a non-frangible test result on a test glass-based article in the form of an example IOX article 10. As shown in FIG. 14A, the test area is a square that is centered at the impact point 530, where the length of a side of the square a is 2 inches. The non-frangible sample shown in FIG. 14A includes three fragments 542, and two crack branches 540 and a single bifurcation 550. Thus, the non-frangible IOX article 10 shown in FIG. 14A contains less than 4 fragments having a largest dimension of at least 1 mm and the number of bifurcations is less than or equal to the number of crack branches. As utilized herein, a crack branch originates at the impact point 530, and a fragment is considered to be within the test area if any part of the fragment extends into the test area.

While coatings, adhesive layers, and the like may be used in conjunction with the strengthened glass articles described herein, such external restraints are not used in determining the frangibility or frangible behavior of the glass-based articles. In some embodiments, a film that does not impact the fracture behavior of the glass-based article 10 may be applied to the glass-based article prior to the frangibility test to prevent the ejection of fragments from the glass article, increasing safety for the person performing the test.

FIG. 14B depicts a frangible test result on a test glass article in the form of an example IOX article 10. The frangible IOX article 10 includes 5 fragments 542 having a largest dimension of at least 1 mm. The IOX article 10 depicted in FIG. 14B includes 2 crack branches 540 and 3 bifurcations 550, producing more bifurcations than crack branches. Thus, the IOX article 10 depicted in FIG. 14B does not exhibit either four or less fragments or the number of bifurcations being less than or equal to the number of crack branches.

In the frangibility test described herein, the impact is delivered to the surface of the glass-based article with a force that is just sufficient to release the internally stored energy present within the strengthened glass-based article. That is, the point impact force is sufficient to create at least one new crack at the surface of the strengthened glass-based article and extend the crack through the compressive stress CS region (i.e., depth of layer) into the region that is under central tension CT.

It will be apparent to those skilled in the art that various modifications to the preferred embodiments of the disclosure as described herein can be made without departing from the spirit or scope of the disclosure as defined in the appended claims. Thus, the disclosure covers the modifications and variations provided they come within the scope of the appended claims and the equivalents thereto.

What is claimed is:

1. A method of estimating a least one stress-based characteristic of a chemically strengthened article having a refractive index profile with a near-surface spike region and a deep region that define an optical waveguide in a glass-based substrate, comprising:
a) using a prism-coupling system having a light source and a coupling prism, sequentially illuminating the glass-based substrate through the coupling prism with measurement light of different wavelengths to generate reflected light containing TM and TE mode spectra for each measurement wavelength to define a set of TM and TE mode spectra;
b) examining the set of TM and TE mode spectra and identifying a best TM and TE mode spectra of the set of TM and TE mode spectra for providing a most accurate estimate of the at least one stress-based characteristic; and
c) estimating the at least one stress-based characteristic using the best TM and TE mode spectra.

2. A method of estimating a least one stress-based characteristic of a chemically strengthened article having a refractive index profile with a near-surface spike region and a deep region that define an optical waveguide in a glass-based substrate, comprising:
a) using a prism-coupling system having a light source and a coupling prism, sequentially illuminating the glass-based substrate through the coupling prism with broadband measurement light to generate broadband reflected light containing TM and TE mode spectra;
b) sequentially narrow-band filtering either the broadband measurement light or the broadband reflected light to form sequential narrow-band reflected light beams having different center wavelengths;
c) digitally detecting the sequential narrow-band reflected light beams to capture TM and TE mode spectra for each of the sequential narrow-band reflected light beams;
d) examining the set of TM and TE mode spectra to identify a best TM and TE mode spectra of the set of TM and TE mode spectra for providing a most accurate estimate of the at least one stress-based characteristic; and
e) estimating the at least one stress-based characteristic using the best TM and TE mode spectra

3. A prism-coupling system for measuring a stress characteristic of a chemically strengthened ion-exchanged (IOX) article having a near-surface spike region and a deep region formed in a glass-based substrate and that define an optical waveguide, comprising:
a) a coupling prism having an input surface, an output surface and a coupling surface, and wherein the coupling surface interfaces with the waveguide at a substrate upper surface;
b) a light source system that sequentially emits over an input optical path multiple measurement light beams having different measurement wavelengths, wherein the sequentially emitted measurement light beams illuminate the interface through the input surface of the prism, thereby forming sequentially reflected light beams that exit the output surface of the coupling prism and travel over an output optical path, wherein the sequentially reflected light beams defines respective transverse magnetic (TM) mode spectrum and a transverse electric (TE) mode spectrum each having a different one of the measurement wavelengths;
c) a photodetector system arranged to receive the sequentially reflected light beams and detect the TM and TE mode spectra for each of the measurement wavelengths to form a set of TM and TE mode spectra;
d) a controller configured to perform the acts of:
a. processing the set of TM and TE mode spectra to identify a best TM and TE mode spectra of the set of TM and TE mode spectra for providing a most accurate estimate of the at least one stress-based characteristic; and
b. estimating the at least one stress-based characteristic using the best TM and TE mode spectra.

4. The prism-coupling system according to claim 3, wherein the light source system comprises a light source device that operably supports multiple light-emitting elements having different measurement wavelengths.

5. The prism-coupling system according to claim 4, wherein the light source device is mechanically connected to a motion control system that moves the light source device so that the light-emitting elements sequentially emit measurement light of the different measurement wavelengths over the input optical path.

6. The prism-coupling system according to claim 5, wherein the motion control system comprises a linear actuator connected to the light source by a drive shaft.

7. The prism-coupling system according to claim 3, wherein the sequentially emitted measurement light beams each has an optical bandwidth and further comprising multiple narrow-band optical filters, wherein the optical filters are supported in a support frame so that each of the light-emitting elements of the multiple light-emitting elements is optically aligned with one of the optical filters to reduce the optical bandwidth of the sequentially emitted measurement light beams.

8. The prism-coupling system according to claim 3, wherein the light source comprises a broadband light-emitting element that emits broadband light, and further comprising an array of optical filters each having a different central wavelength, wherein the optical filters are supported by a movable support frame so that the optical filters can be sequentially inserted into the broadband light to generate the sequential measurement light beams having the different measurement wavelengths.

9. The prism-coupling system according to claim 8, wherein the movable support frame comprises a rotatable filter member.

10. The prism-coupling system according to claim 8, wherein the movable support frame is operably attached to a linear actuator configured to translate the support frame to sequentially insert the optical filters into the broadband light.

11. The prism-coupling system according to claim 3, wherein the sequentially emitted measurement light beams each has an optical bandwidth of less than 10 nm.

12. The prism-coupling system according to claim 3, wherein the different measurement wavelengths consist of three different measurement wavelengths.

13. The prism-coupling system according to claim 3, wherein the different measurement wavelengths fall within the wavelength range from 350 nm to 850 nm.

14. A prism-coupling system for measuring a stress characteristic of a chemically strengthened ion-exchanged (IOX) article having a near-surface spike region and a deep region formed in a glass-based substrate and that define an optical waveguide, comprising:
a) a coupling prism having an input surface, an output surface and a coupling surface, and wherein the coupling surface interfaces with the waveguide at a substrate upper surface;
b) a light source system that sequentially emits over an input optical path a broadband light beam that illuminates the interface through the input surface of the prism, thereby forming a reflected light beam that exit the output surface of the coupling prism and travel over an output optical path, wherein the reflected light beam defines a transverse magnetic (TM) mode spectrum and a transverse electric (TE) mode spectrum;
c) an optical filter system configured to sequentially insert optical filters having different narrow-band wavelength transmissions into either the input optical path or the output optical path to define sequentially reflected light beams each having a different measurement wavelength;
d) a photodetector system arranged to receive the sequential reflected light beams and detect the TM and TE mode spectra for each of the measurement wavelengths to form a set of TM and TE mode spectra;
e) a controller configured to perform the acts of:
a. identifying a best TM and TE mode spectra of the set of TM and TE mode spectra for providing a most accurate estimate of the at least one stress-based characteristic; and
b. estimating the at least one stress-based characteristic using the best TM and TE mode spectra.

15. The prism-coupling system according to claim 14, wherein the optical filter system comprises a movable filter member mechanically connected to a drive motor configured to move the movable filter member.

16. The prism-coupling system according to claim 15, further comprising a detection system for detecting a position of the movable filter member.

17. The prism-coupling system according to claim 15, wherein the moveable filter member comprises a rotatable filter wheel.

18. The prism-coupling system according to claim 14, wherein said identifying the best TM and TE mode spectra comprises performing a fringe count to determine an integer part of the fringe count and a fractional part of the fringe count and selecting the TM and TE mode spectra based on the fractional part of the fringe count falling within a select range.

19. The prism-coupling system according to claim 14, wherein the sequentially reflected light beams each has a wavelength band of less than 10 nm.

20. The prism-coupling system according to claim 14, wherein the different measurement wavelengths fall within the wavelength range from 350 nm to 850 nm.
